(19) United States
(12) Patent Application Publication
(10) Pub. No.: US 2023/0355652 A1
(43) Pub. Date: Nov. 9, 2023

(54) FETAL STROMAL CELL DERIVED EXOSOMES FOR TISSUE REPAIR

(71) Applicant: BioCardia, Inc., Sunnyvale, CA (US)

(72) Inventor: Ian McNiece, Coral Gables, FL (US)

(21) Appl. No.: 18/343,698

(22) Filed: Jun. 28, 2023

Related U.S. Application Data
(63) Continuation of application No. PCT/US21/65586, filed on Dec. 29, 2021.
(60) Provisional application No. 63/133,599, filed on Jan. 4, 2021.

(51) Int. Cl. A61K 31/7105; A61K 35/34

(52) U.S. Cl. CPC A61K 31/7105; A61K 35/34

(57) ABSTRACT

Cardiac stromal cells produce exosomes containing micro RNAs including: miR1, miR133a, and miR206. These micro RNAs have pivotal roles in development and physiology of muscle tissues including the heart. Exosomes containing these micro RNAs are delivered to result in regeneration of ischemic heart tissue.

### CROSS-REFERENCE

This application is a continuation of PCT No. PCT/US21/65586 (Attorney Docket No. 29181-716.601), filed Dec. 29, 2021, which claims the benefit of priority to U.S. Provisional Patent Application No. 63/133,599 (Attorney Docket No. 29181-716.101), filed Jan. 4, 2021, the entire content of each of which is incorporated herein by reference.

### BACKGROUND

### 1. Field of the Invention

The homeostasis of cellular content is an ongoing process in most tissue, representing a balance between cell death and new cell production. One of the best studied systems in the body is the hematopoietic system which requires ongoing blood cell production due to blood cell turnover. The bone marrow (BM) is the principal site for blood cell formation in humans. In normal adults, the body produces about 2.5 billion red blood cells (RBC), 2.5 billion platelets and 10 billion granulocytes per kilogram of body weight per day [1]. The production of mature blood cells is a continual process that is the result of proliferation and differentiation of stem cells, committed progenitor cells and differentiated cells. Within these three stages, extensive expansion of cell numbers occurs through cell division. A single stem cell has been proposed to be capable of more than 50 cell divisions or doublings and has the capacity to generate up to 10¹⁵ cells, or sufficient cells for up to 60 years [2]. The proliferation and differentiation of cells is controlled by a group of proteins called hematopoietic growth factors (HGFs). The GFs and other cytokines are produced in part by stromal cells of the BM microenvironment. In addition, hematopoietic stem cells (HSC) reside in the BM in close proximity to stromal cells which provide the “stem cell niche”. Deficiencies in the microenvironment at a cellular or molecular level result in abnormal cell production resulting in anemia, leukemia, or embryonic lethality [3].

Studies have demonstrated the existence of tissue specific stem cells in most if not all tissues and organs of the body. It has been proposed that stromal cells make up a “BM-like” stem cell niche in tissues, such as the heart.

### 2. Listing of Relevant Art

Relevant patents and patent publications include U.S. Pat. No. 9,884,076; US2020/0323924; US2020/0197535; and US2015/0203844.

### SUMMARY

In a first aspect, the present disclosure provides a composition comprising an exosome derived from a fetal cardiac stromal cell, said exosome containing one or more of miR1, miR133a, and miR206, often containing at least two of miR1, miR133a, and miR206, and frequently containing all three of miR1, miR133a, and miR206.

In a second aspect, the present disclosure provides a method of treating a patient suffering from a cardiac disease, disorder, or injury. Said method comprising administering to the patient's cardiac tissue the exosome of derived from a fetal cardiac stromal cell, said exosome containing one or more of miR1, miR133a, and miR206, often containing at least two of miR1, miR133a, and miR206, and frequently containing all three of miR1, miR133a, and miR206. the patient's cardiac tissue. Often, patient's cardiac tissue comprises infarcted myocardial tissue.

In a third aspect, the present disclosure provides a method of treating a cardiac disease, disorder, or injury in a subject. The method comprises administering to a patient in need thereof an effective amount of a composition comprising an agent comprising an exosome containing one or more of miR1, miR133a, and miR206, often containing at least two of miR1, miR133a, and miR206, and frequently containing all three of miR1, miR133a, and miR206. The cardiac disease, disorder, or injury is typically myocardial infarction. In specific instances, the composition may be administered to the patient by a route selected from the group consisting of local, topical, subcutaneous, intravenous, oral, intramuscular, and combinations thereof. In some instances, the composition may be administered to the patient by myocardial injection. The composition may be provided in a solution suitable for injection such as Plasma-Lyte.

In a fourth aspect, the present disclosure provides method for producing a therapeutic composition comprising harvesting a population of exosomes from fetal cardiac stromal cell, where the exosomes containing one or more of miR1, miR133a, and miR206, often containing at least two of miR1, miR133a, and miR206, and frequently containing all three of miR1, miR133a, and miR206.

In a fifth aspect, the present disclosure provides a composition comprising an exosome derived from a fetal stromal cell. The exosome may contain two or more of miR which are more than two fold TPM (transcripts per million) in the fetal stromal cell than in an adult mesenchymal stem cell. The exosome may be targeted for a reparative therapeutic indication towards a degenerative tissue disease that is specific to the tissue from which said fetal stromal cell is derived.

The exosomes may be derived from fetal cardiac stromal cells. The two or more miR are selected from the set of miR1, miR133a, miR206, hsa-miR-146a-5p, hsa-miR-490-3p, hsa-miR-9-5p, hsa-miR-3117-3p, hsa-miR-4521, hsa-miR-412-5p, hsa-miR-541-3p, hsa-miR-6724-5p, hsa-miR-182-5p, hsa-miR-454-5p, hsa-miR-206, hsa-miR-584-5p, hsa-miR-7706, hsa-miR-3177-3p, hsa-miR-410-5p, hsa-miR-541-5p, hsa-miR-3175, hsa-miR-204-5p, hsa-miR-3661, hsa-miR-302a-5p, hsa-miR-4661-5p, hsa-miR-543, hsa-miR-103a-2-5p, hsa-miR-3176, hsa-miR-433-5p, hsa-miR-10a-5p, hsa-miR-6753-3p, hsa-miR-330-5p, hsa-miR-11401, hsa-miR-582-3p, hsa-miR-2355-3p, hsa-miR-6511b-5p, hsa-miR-494-5p, hsa-miR-548k, hsa-miR-200a-3p, hsa-miR-744-3p, hsa-miR-487a-3p, hsa-miR-4665-5p, hsa-miR-598-3p, hsa-miR-548ao-3p, hsa-miR-301a-5p, hsa-miR-940, hsa-miR-323a-5p, hsa-miR-1228-5p, hsa-miR-760, hsa-miR-495-3p hsa-miR-937-3p, hsa-miR-4684-3p, hsa-miR-337-3p, hsa-miR-3138, hsa-miR-433-3p, hsa-miR-487b-5p, hsa-miR-2682-5p, hsa-miR-6732-3p, hsa-miR-3167, hsa-miR-3187-3p, hsa-miR-219a-1-3p, hsa-miR-18a-3p, hsa-miR-1343-3p, hsa-miR-98-5p, hsa-miR-191-3p, hsa-miR-33a-3p, hsa-miR-143-3p, hsa-miR-432-5p, hsa-miR-548ay-3p, hsa-miR-1307-3p, hsa-miR-8485, hsa-miR-487a-5p, hsa-miR-451a, hsa-miR-33b-3p, hsa-miR-3155b, hsa-miR-380-5p, hsa-miR-10401-3p, hsa-miR-539-5p hsa-miR-323b-3p, hsa-miR-3605-3p, hsa-miR-3064-5p, hsa-miR-3691-5p, hsa-miR-6827-5p, hsa-miR-487b-3p, hsa-miR-3074-5p, hsa-miR-100-5p, hsa-miR-24-3p, hsa-miR-149-5p, hsa-miR-1909-3p, hsa-miR-3675-3p, hsa-miR-323a-3p, hsa-miR-129-5p, hsa-miR-187-3p, hsa-miR-431-5p, and hsa-miR-200a-5p. The two or more miR may be selected to treat a clinical indication selected from the set of heart failure, myocardial infarction, and chronic myocardial ischemia.

The exosomes may be derived from fetal liver stromal cells to treat liver disease that results from drug toxicity, alcoholism, Hepatitis B, or Hepatitis C.

The exosomes may be derived from fetal brain stromal cells to treat stroke or Parkinson's disease.

The exosomes may be derived from fetal kidney stromal cells to treat kidney failure.

The exosomes may be derived from fetal skin stromal cells to treat psoriasis or other diseases and conditions of the skin.

The exosomes may be derived from fetal lung stromal cells and used to treat asthma and emphysema.

The exosomes may be derived from fetal pancreas stromal cells and used to treat type 1 diabetes.

The exosomes may be derived from fetal pancreas stromal cells and used to treat type 2 diabetes.

In some cases, the exosome in the composition contains three or more of miR which are more than two fold TPM in the fetal stromal cell than in the adult mesenchymal stem cell. The exosome may be targeted for a reparative therapeutic indication towards a degenerative tissue disease that is specific to the tissue from which said fetal stromal cell is derived.

In some cases, the exosome contains four or more of miR which are more than two fold TPM in the fetal stromal cell than in the adult mesenchymal stem cell. The exosome may be targeted for a reparative therapeutic indication towards a degenerative tissue disease that is specific to the tissue from which said fetal stromal cell is derived.

In some cases, the exosome contains two or more of miR which are more than three fold TPM in the fetal stromal cell than in the adult mesenchymal stem cell. The exosome may be targeted for a reparative therapeutic indication towards a degenerative tissue disease that is specific to the tissue from which said fetal stromal cell is derived.

The present disclosure may provide methods of treating a patient suffering from the aforementioned diseases or degenerative tissue disease by administering the composition comprising the exosome to the corresponding tissue of the patient.

While reference is made herein to exosomes from fetal cardiac stromal cells and to treatment of cardiac disease, various aspects of the present are applicable to exosomes from other cell types and sources and to other diseases. Other tissue specific fetal stromal cells from other fetal tissues can be obtained and expanded and used to develop therapeutic cell and cell derived therapies. Such therapies may be targeted towards therapeutic applications targeted to impact those tissues. These tissues may include, for example, skin, lung, liver, brain, kidney, spleen, thymus, pancreas, muscle, umbilical cord, umbilical cord blood, amnion, placenta, and amniotic fluid, to name a few.

Diseases to be treated include but are not limited to psoriasis, asthma, emphysema, liver damage, stroke, kidney failure, heart failure, and diabetes. Liver damage can be caused by drug toxicity, alcoholism, hepatitis B, hepatitis C, and other causes. Heart failure may be ischemic or nonischemic in etiology and may be characterized as systolic or diastolic in nature. Stroke may be ischemic or hemorrhagic. Diabetes may include type I or type 2 diabetes.

Cell therapies may include, for example, cells, cells in a matrix such as collagen, fibronectin, fibrin, and cell aggregates such as spheroids and sheets, to name a few. Cell derived therapies may include, for example, exosomes and miRNAs.

### BRIEF DESCRIPTION OF THE DRAWINGS

FIG. 1 shows three graphs showing cellular expression of CD73, CD90, and CD105 in cardiac stromal cells (CStrCs) in comparison to isotypes.

FIG. 2 shows a graph comparing the expression of miR-1, miR-133a, and miR-206, respectively, in CStrCs to expression in BM MSCs (bone marrow mesenchymal stem cells).

### DETAILED DESCRIPTION

### The Bone Marrow Microenvironment

The BM from a wide range of mammalian species contains precursor cells that generate adherent colonies of stromal cells in vitro. The BM stroma represents the non-hematopoietic connective tissue elements that provide a system of structural support for developing hematopoietic cells. The complex cellular composition of marrow stromal tissue comprises a heterogenous population of cells including reticular cells, adipocytes, osteogenic cells near bone surfaces, vascular endothelial cells, smooth muscle cells in vessel walls, and macrophages [4-7].

The concept that adult hematopoiesis occurs in a stromal microenvironment within the BM was first proposed by Dexter and colleagues, leading to the establishment of the long-term BM culture (LTMC). These studies demonstrated that an adherent stromal-like culture could support maintenance of hematopoietic stem cells (HSC) [8]. Mesenchymal stem cells (MSC) represent the major stromal cell population in the BM.

Mesenchymal stem cells (MSC) MSCs were recognized by Friedenstein who isolated cells from guinea pig bone marrow which were adherent in culture and which differentiated into bone [9]. Surface antigens have been reported for identification and phenotyping of human MSCs [10-12]. Although MSCs are rare, representing approximately 0.01% of the bone marrow mononuclear cell fraction, they have attractive features for therapy, including the ability to expand many log-fold in vitro, and unique immune characteristics allowing their use as an allogeneic graft. They are typically isolated based upon adherence to standard tissue culture flasks. Low density BM mononuclear cells (MNCs) are placed into culture in basal media plus FCS (typically 20%) and after 2 to 3 days adherent cells can be visualized on the surface of the flask. The non-adherent cells are removed at this time and fresh media added until a confluent adherent layer forms. The MSC are harvested by treatment with trypsin and further passaged expanding the number of MSC. A number of different cell populations have been isolated using different culture conditions however, the morphology of these cells is very similar. Phenotypical characterization of MSC has been performed by many groups and a standard criteria has been proposed by the International Society of Cellular Therapy (ISCT) [13]. The minimal criteria proposed to define human MSC by the Mesenchymal and Tissue Stem Cell Committee of the ISCT consists of the following: 1) the MSC must be plastic-adherent when maintained in standard culture conditions; 2) MSC must express CD105, CD73 and CD90, and lack expression of CD45, CD34, CD14 or CD11b, CD79 alpha or CD19 and HLA-DR surface molecules; and 3) MSC must differentiate into osteoblasts, adipocytes and chondrocytes in vitro [13].

A standard in vitro assay for MSC is the colony-forming unit fibroblast (CFU-F) assay [14]. BM MNCs are plated at low density and colonies of fibroblasts develop attached on the surface of the culture dish. Based upon the results of this assay, the frequency of MSC precursor cells is one in 10⁴ to 10⁵ BM MNC. The frequency is highly variable between individuals and the number of MSC has been shown to be decreased in older people. Other studies have demonstrated that MSC precursors can be isolated based upon surface antigen expression. Antibodies to CD271 and Stro-1 have been used to enrich MSC precursors. CD271, also known as low affinity nerve growth factor receptor (LNGFR) or p75NTR, belongs to the low affinity neurotrophin receptor and the tumor necrosis factor receptor superfamily. Selection of CD271+ cells from human BM enriches CFU-F and MSC are preferentially selected in the CD271+ fraction compared to the CD271− fraction [10,11]. Similarly, isolation of Stro-1+ cells from BM MNC results in enrichment of CFU-F in the Stro-1+ fraction compared to the Stro-1-fraction [12].

Immunologic properties of MSCs. MSCs are ideal candidates for allogeneic transplantation because they show minimal MHC class II and ICAM expression and lack B-7 costimulatory molecules necessary for T-cell mediated immune responses [15-17]. Indeed, MSCs do not stimulate a proliferative response from alloreactive T-cells even when the MSCs have differentiated into other lineages or are exposed to proinflammatory cytokines [17]. As previously reviewed [18], MSCs have significant immunomodulatory effects, inhibiting T-cell proliferation [19], prolonging skin allograft survival [20], and decreasing graft-versus-host disease (GVHD) [21]. Recently human MSCs were shown to alter the cytokine secretion profile of dendritic cells, T cells, and natural killer cells in vitro, inhibiting secretion of proinflammatory cytokines (e.g. TNF-a, IFN-γ) and increasing expression of suppressive cytokines (e.g. IL-10), possibly via a prostaglandin E2 mediated pathway [22]. In vivo studies of the fate of MSCs have shown that, when transplanted into fetal sheep, human MSCs engraft, undergo site-specific differentiation into various cell types, including myocytes and cardiomyocytes and persist in multiple tissues for as long as 13 months after transplantation in non-immunosuppressed immunocompetent hosts [23]. Further, in vivo studies using rodents, dogs, goats, and baboons demonstrate that allogeneic MSCs can be engrafted into these species without stimulating systemic alloantibody production or eliciting a proliferative response from recipient lymphocytes [24-27]. These properties present MSC as a promising source of allogeneic cells for tissue repair.

The Stem Cell Niche. The control of proliferation and differentiation of a number of types of stem cells (SC) occurs in the micro environmental niche or the stem cell niche. Hematopoietic stem cells (HSC) have been studied in detail and shown to reside in the bone marrow in association with stromal cells which make up the hematopoietic microenvironment [28]. The stroma consists of several cell populations including mesenchymal stem cells (MSC), fibroblasts and adventicular reticulocytes [29]. HSC exist in a quiescent state in close relationship with the stromal cells in the bone marrow. These stromal cells produce a number of cytokines and growth factors that are either secreted or expressed as membrane bound proteins and these cytokines and growth factors control the differentiation and proliferation of the HSC. In vitro, MSC have been shown to support the proliferation and differentiation of HSC, generating committed hematopoietic progenitor cells over a six-week period [8]. If the microenvironment is compromised, such as in patients who receive multiple rounds of high dose chemotherapy regimens, normal homeostasis is disrupted and deficiencies in blood cells occurs.

Stromal Cells in Cardiac Tissue. The extracellular matrix (ECM) of cardiac tissue provides elasticity and mechanical strength. The cardiac ECM is composed of a number of cells including cardiac fibroblasts, mesenchymal cells, fibronectin, and other matrix proteins [30-32]. We have isolated several stromal cell populations from human heart tissue which are positive for CD105, CD90, and CD73 but negative for CD34 and CD45, which is consistent with the phenotype of BM derived MSC. Given the homeostatic role of MSC in regulation of HSC it is highly likely that cardiac stromal cells play a regulatory role in the control of proliferation and differentiation of cardiac stem and progenitor cells (CSC and CPC). This role could be performed through the secretion of a range of growth factors and cytokines.

MI results in ischemic damage which results in cell death of not only cardiomyocytes but also fibroblasts and most likely stromal cells. Even with migration of viable CSCs and CPCs to the ischemic tissue, the lack of stromal elements would result in the failure of the CSCs and CPCs to proliferate and differentiate, hence failure of remodeling. Along with the recent identification of cardiac stem cells in heart tissue, this offers insights into the biology of ischemic heart damage. Patients with an MI have ischemic tissue which fails to regenerate and we propose that this is in part due to destruction of cardiac stromal cells.

MSC derived from BM cells have been evaluated for cardiac regenerative therapy [33] and as presented above, offer advantages over other sources of stem cells because of their availability, immunologic properties, and record of safety and efficacy. Studies of MSC engraftment in rodent and swine models of myocardial infarction demonstrate: 1) functional benefit in post-myocardial infarction (MI) recovery with administration 2) evidence of neoangiogenesis at the site of the infarct 3) decrease in collagen deposition in the region of the scar 4) some evidence of cells expressing contractile and sarcomeric proteins but lacking true sarcomeric functional organization. Administration of autologous or allogeneic human MSCs to cardiovascular patients has been performed in several clinical studies to date, all in the post-myocardial infarction (MI) setting. The MSC have been administered via the intracoronary route (IC), via peripheral intravenous (IV) injection or direct injection into the cardiac tissue with surgery.

### Cardiac Derived Stromal Cells (CStrCs)

Isolation of MSC. Bone marrow cells were purchased from Allcells Inc. (Emeryville, CA) who obtained the BM aspirates from normal donors under appropriate IRB approvals. The MNC fraction was isolated by ficol separation and MSC were grown to confluency in T162 cm² tissue culture flasks (Corning, Acton, MA) in alpha MEM plus 20% FCS.

Isolation of CStrCs from human heart tissue. Human fetal heart tissue was obtained with appropriate consent and IRB approval from aborted fetuses (15-22 weeks of gestation) from Advanced Biosciences Resources Inc (Alameda, CA). The heart tissue was washed and dissected into small pieces and digested using collagenase IV (0.5% w/v; Invitrogen, Carlsbad, CA) for 5 minutes. The cell suspension was passed through a cell strainer and counted using Trypan Blue for viability. The cells were cultured in T162 cm² tissue culture flasks in alpha MEM plus 20% FCS with twice weekly media exchange. Adherent cells developed within two weeks and were passaged using trypsin treatment when confluent.

Micro Array Analysis: Total RNA Extraction, microarray hybridization, and data analysis. RNA was extracted from three independent cultures of each of the stromal cell lines from adult human bone marrow and fetal heart. Total RNA was isolated using TRIzol reagent (Invitrogen Life Technologies, Carlsbad, CA, USA) according to the manufacturer's instruction and additionally purified with RNeasy Mini Kit (Qiagen, Cat #74106). RNA was quantified with a Nanodrop 8000 Spectrophotometer (Thermo Scientific, Wilmington) and its quality was examined with a Bioanalyzer 2100 using the RNA 6000 Nano kit (Agilent, Santa Clara, CA). Biotinylated cRNA was prepared using the Illumina TotalPrep RNA Amplification Kit (Ambion, Inc., Austin, TX) according to the manufacturer's instructions starting with 400 ng total RNA. Successful cRNA generation was checked using the Bioanalyzer 2100. Samples were added to the Beadchip after randomization using the randomized block design to reduce batch effects. Hybridization to the Sentrix Human-6 Expression BeadChip (Illumina, Inc., San Diego, CA), washing and scanning were performed according to the Illumina BeadStation 500 manual (revision C). The resulting microarray data was analyzed using Illumina Beadstudio software.

TaqMan Real-time PCR microRNA array. Total RNA was isolated as described above. RNA quality was assessed with a Nanodrop 8000 Spectrophotometer (Thermo Scientific, Wilmington) and RNA integrity and presence of the small RNA fraction was determined using a Bioanalyzer 2100 (Agilent, Santa Clara). 60 ng of total RNA was reverse transcribed using the human megaplex pool A and B primers and the Taqman miRNA reverse transcription kit (Applied Biosystems, Foster City) according to the manufacturer's instructions. Each sample was pre-amplified for 12 cycles using human pool A and B pre-amplification primers and the Taqman PreAmp Master Mix (Applied Biosystems) according to the manufacturer's instructions. For each sample the pre-amplification reactions A and B were diluted and each reaction was combined with Taqman Gene-Expression Master Mix (Applied Biosystems) split in 8 aliquots and each aliquot added to one of the eight sample ports of the Human miRNA Taqman array A or B, respectively. Each of the ports of the Taqman array feeds 48 reaction vessels holding individual miRNA assays. Human miRNA Taqman array A and B hold 667 different miRNA target and 4 miRNA reference assays. The real-time PCR reactions were run according to the manufacturer's instructions. RealTime Statminer Software (Integromics, Philadelphia) was used to analyze the data. The reference miRNA assays included on the Taqman arrays did not pass the expression stability test. Therefore reference miRNA assays were chosen based on expression stability between different samples using the GeNorm algorithm [1].

Quantitative-PCR. Real-time PCR for miR-1, miR-133a, and miR206 was performed on the CStrCs and BM MSC. The single tube TaqMan MicroRNA Assay was used. All reagents, primers and probes were obtained from Applied Biosystems (Applied Biosystems, Foster City, CA). RNU6B was used as a normalizer. One nanogram of RNA per sample was used for the assays. All RT reactions, including no-template (no cDNA) controls and minus controls (no reverse transcriptase), were run in a GeneAmp PCR 9700 Thermocycler (Applied Biosystems). Gene expression levels were quantified using the ABI Prism 7900HT Sequence detection system (Applied Biosystems). Comparative real-time PCR was performed in triplicate. Expression of the microRNAs was calculated utilizing the comparative Ct method and compared with t test. P<0.05 was considered statistically significant.

Flow Cytometric Analysis. Cells were analyzed for phenotypic expression of surface proteins with a minimum of 50,000 events collected in a list mode file format by flow cytometry (FACS Vantage, Becton-Dickinson). Aliquots of cells were also stained with isotype control antibodies.

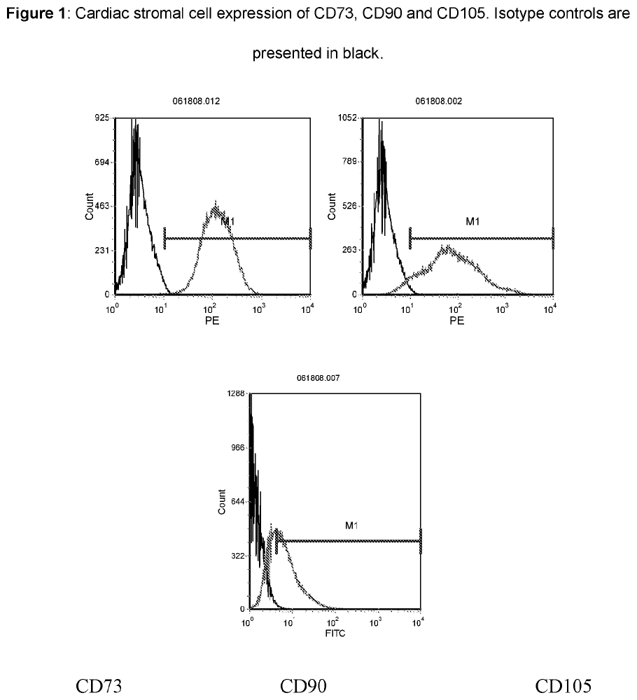

Characterization of Cardiac Stromal Cells. More than 20 different CStrC lines have been isolated from human heart tissue. In all cases, flow cytometry analysis demonstrated an equivalent phenotype to BM MSC with positive staining for CD105, CD73, and CD90 (FIG. 1) and negative for CD45 and CD34. BM MSC were negative for CXCR4, however, 5 to 10% of CStrCs were positive for CXCR4 expression.

The CStrCs formed CFU-F colonies when plated in vitro, with a median of 26 (range 17 to 32) CFU-F per 100 cells plated. In contrast, BM MSCs contained fewer CFU-F (p<0.01), forming only 3 (range 2 to 5) CFU-F per 100 cells plated. These data suggest that CStrCs have a higher proliferative potential than BM MSCs and we have confirmed this with continued passage of CStrCs resulting in shorter time periods from initiation to confluency through passages P2 to P10.

Array Analysis. The BM MSC and CStrCs were culture expanded and then RNA prepared for micro array analysis. We have performed a global gene array analysis and a micro RNA array. This analysis demonstrated distinct gene patterns between these two sources of stromal cells. In particular, our analysis has demonstrated distinct cytokine and cytokine receptor patterns (Table 1). These data are suggestive of stromal cells in different tissues secreting differing cytokines and expressing different cytokine receptors consistent with local control of tissue specific stem cells and progenitor cells through cytokines. CStrCs also expressed increased gene levels of cell adhesion molecules and focal adhesion molecules (Table 2 and 3) with an increase in a number of genes associated with endothelial, vascular and muscle cells. The increased expression of myosin genes and laminin alpha 5 would be consistent with cardiac expression compared to bone marrow derived cells.

### Micro RNA Arrays

There were 26 miRs up regulated and 6 down regulated by more than two-fold in CStrCs compared to BM MSC (Table 4). Three (miR-1, miR-133a, and miR-206) of the up regulated miRs have functions in myogenesis and cardiomyocyte development [10,11] and two (miR-20a and miR-26a) have function in stem cell differentiation [12,13]. Of particular note was miR-206, which was up regulated by more than one thousand-fold in CStrCs. miR-206 has been reported as a muscle specific miR that promotes muscle differentiation and regulates connexin 43 expression during skeletal muscle development [14,15]. In addition, miR-1 and miR-206 has been reported to be upregulated in the heart following myocardial infarction compared to normal heart [16].

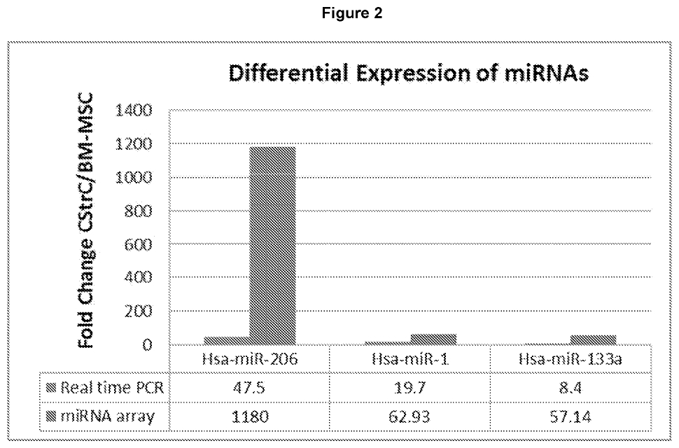

Therefore, we further evaluated the levels of expression of miR-1, 133a, and 206 in CStrCs by q-PCR. There was a 20-fold, 8 fold and 46 fold higher expression of miR-1, miR-133a, and miR-206 respectively, in CStrCs compared to BM MSCs (FIG. 2).

The microenvironment is a key component of tissue homeostasis controlling proliferation and differentiation of stem cells and providing key signals, such as growth factors for control of function of mature cells. In the bone marrow, MSCs are the major micro environmental cell population and have been shown to support hematopoietic stem cells. The role of stromal cells in other tissues remains unclear. Here we present data that demonstrates that heart derived stromal cells have a similar phenotype to BM derived MSCs, however, the gene and micro RNA expression profiles are distinct. The gene profiles of growth factors and cytokines differ between the two cell populations. Also, the micro array data for micro RNA demonstrate that the CStrCs express cardiac related miRs at much higher levels than BM derived MSCs. The expression of miR-1, miR-133a, and miR-206 in CStrCs suggest that miRs play an important role in the function of stromal cells in the heart. These miRs are necessary for proper skeletal and cardiac muscle development and function, and have a significant influence on multiple myopathies, such as hypertophy, dystrophy and conduction defects.

TABLE 1
Cytokines and Cytokine Receptors Expressed at 2 fold or Higher
Levels in Cardiac Stromal Cells Compared to Bone Marrow MSC.

| | |
|---|---|
| IL10RB | Interleukin 10 receptor, beta |
| TNFRSF11B | Tumor necrosis factor receptor superfamily, member 11b (osteoprotegerin) |
| TGFBR2 | Transforming growth factor, beta receptor II (70/80 kDa) |
| TNFRSF12A | Tumor necrosis factor receptor superfamily, member 12A |
| IFNGR2 | Interferon gamma receptor 2 (interferon gamma transducer 1) |
| FAS | Fas (TNF receptor superfamily, member 6) |
| PDGFRA | Platelet-derived growth factor receptor, alpha polypeptide |
| CXCL16 | Chemokine (C-X-C motif) ligand 16 |
| GHR | Growth hormone receptor |
| VEGFC | Vascular endothelial growth factor C |
| CSF1R | Colony stimulating factor 1 receptor, formerly McDonough feline sarcoma viral |
| | (v-fms) oncogene homolog |
| TNFRSF14 | Tumor necrosis factor receptor superfamily, member 14 (herpesvirus entry |
| | mediator) |
| CCL26 | Chemokine (C-C motif) ligand 26 |
| MET | Met proto-oncogene (hepatocyte growth factor receptor) |
| IL11RA | Interleukin 11 receptor, alpha |
| LEPR | Leptin receptor |

TABLE 2
Cell Adhesion Molecules Expressed at 2 fold or Higher Levels
in Cardiac Stromal Cells Compared to Bone Marrow MSC

| | |
|---|---|
| NRXN2 | neurexin 2 |
| ITGB2 | integrin, beta 2 (complement component 3 receptor 3 and 4 subunit) |
| VCAM1 | Vascular cell adhesion molecule 1 |
| CD99 | CD99 molecule |
| HLA-F | Major histocompatibility complex, class I, F |
| HLA-E | Major histocompatibility complex, class I, E |
| ICAM3 | Intercellular adhesion molecule 3 |
| ICAM2 | Intercellular adhesion molecule 2 |
| HLA-DMA | Major histocompatibility complex, class II, DM alpha |
| NFASC | neurofascin homolog (chicken) |

TABLE 3
Focal Adhesion Molecules Expressed at 2 fold or Higher Levels
in Cardiac Stromal Cells Compared to Bone Marrow MS

| | |
|---|---|
| PIK3CD | Phosphoinositide-3-kinase, catalytic, delta polypeptide |
| MYLK | Myosin, light polypeptide kinase |
| CCND2 | Cyclin D2 |
| LAMA5 | Laminin, alpha 5 |
| MYLPF | Fast skeletal myosin light chain 2 |
| ITGA1 | Integrin, alpha 1 |
| TNC | Tenascin C (hexabrachion) |

TABLE 4
Micro RNAs Expressed at Higher or Lower Levels in
Cardiac Stromal Cells Compared to Bone Marrow MSC.

| miR | Fold Up |
|---|---|
| miR-1 | 63 |
| miR-10a | 11 |
| miR-15a | 3 |
| miR-15b | 6 |
| miR-16-1 | 16 |
| miR-18a | 24 |
| miR-19b-1 | 15 |
| miR-20a | 16 |
| miR-26a-2 | 7 |
| miR-33a | 26 |
| miR-92a-1 | 137 |
| miR-133a | 57 |
| miR-206 | 1181 |
| miR-374a | 4 |
| miR-411 | 2 |
| miR-424 | 10 |
| miR-450a | 2 |
| miR-450b-5p | 37 |
| miR-454 | 3 |
| miR-503 | 35 |
| miR-516a-3p | 2 |
| miR-542-3p | 3 |
| miR-551b | 5 |
| miR-589 | 5 |
| miR-770-5p | 2 |
| miR-935 | 213 |

| miR | Fold Down |
|---|---|
| miR-10b | 9 |
| miR-335 | 13 |
| miR-451 | 13 |
| miR-479 | 8 |
| miR-628-3p | 9 |
| miR-768-3p | 2 |

Genes expressed at 2-fold higher (Up regulated) or lower (Down regulated) levels in CStrCs compared to BM MSC are presented.

### Exosomes

Exosomes are extracellular vesicles that are produced in the endosomal compartment of most eukaryotic cells. The multivesicular body is an endosome defined by intraluminal vesicles that bud inward into the endosomal lumen. If the MVB fuses with the cell surface, these ILVs are released as exosomes. In multicellular organisms, exosomes and other EVs are present in tissues and can also be found in biological fluids including blood, urine, and cerebrospinal fluid. They are also released in vitro by cultured cells into their growth medium. Since the size of exosomes is limited by that of the parent MVB, exosomes are generally thought to be smaller than most other EVs, from about 30 to several hundred nanometers in diameter: around the same size as many lipoproteins but much smaller than cells. Compared with EVs in general, it is unclear whether exosomes have unique characteristics or functions or can be separated or distinguished effectively from other EVs. EVs including exosomes carry markers of cells of origin and have specialized functions in physiological processes, from coagulation and intercellular signaling to waste management. Consequently, there is a growing interest in clinical applications of EVs as biomarkers and therapies.

Scientists are actively researching the role that exosomes may play in cell-to-cell signaling, hypothesizing that because exosomes can merge with and release their contents into cells that are distant from their cell of origin (see membrane vesicle trafficking), they may influence processes in the recipient cell [39]. For example, RNA that is shuttled from one cell to another, known as “exosomal shuttle RNA,” could potentially affect protein production in the recipient cell. [17][40] By transferring molecules from one cell to another, exosomes from certain cells of the immune system, such as dendritic cells and B cells, may play a functional role in mediating adaptive immune responses to pathogens and tumors.[15][30]

Conversely, exosome production and content may be influenced by molecular signals received by the cell of origin. As evidence for this hypothesis, tumor cells exposed to hypoxia secrete exosomes with enhanced angiogenic and metastatic potential, suggesting that tumor cells adapt to a hypoxic microenvironment by secreting exosomes to stimulate angiogenesis or facilitate metastasis to more favorable environment. [21] It has recently been shown that exosomal protein content may change during the progression of chronic lymphocytic leukemia. [41]

A study hypothesized that intercellular communication of tumor exosomes could mediate further regions of metastasis for cancer. Hypothetically, exosomes can plant tumor information, such as tainted RNA, into new cells to prepare for cancer to travel to that organ for metastasis. The study found that tumor exosomal communication has the ability to mediate metastasis to different organs. Furthermore, even when tumor cells have a disadvantage for replicating, the information planted at these new regions, organs, can aid in the expansion of organ specific metastasis. [42]

Exosomes carry cargo, which can augment innate immune responses. For example, exosomes derived from Salmonella enterica-infected macrophages but not exosomes from uninfected cells stimulate naive macrophages and dendritic cells to secrete pro-inflammatory cytokines such as TNF-α, RANTES, IL-1ra, MIP-2, CXCL1, MCP-1, sICAM-1, GM-CSF, and G-CSF. Proinflammatory effects of exosomes are partially attributed to lipopolysaccharide, which is encapsulated within exosomes. [43]

Micro RNA 1 (miR1)

MiR1 has pivotal roles in development and physiology of muscle tissues including the heart. [1,2]. MiR-1 is known to be involved in important role in heart diseases such as hypertrophy, myocardial infarction, and arrhythmias. [3][4][5] Studies have shown that MiR-1 is an important regulator of heart adaption after ischemia or ischemic stress and it is upregulated in the remote myocardium of patients with myocardial infarction. [6] Also MiR-1 is downregulated in myocardial infarcted tissue compared to healthy heart tissue.[7] Plasma levels of MiR-1 can be used as a sensitive biomarker for myocardial infarction. [8]

Micro RNA 133 (miR133) mir-133 is a type of non-coding RNA called a microRNA that was first experimentally characterized in mice. [1] Homologues have since been discovered in several other species including invertebrates such as the fruit fly Drosophila melanogaster. Each species often encodes multiple microRNAs with identical or similar mature sequence. For example, in the human genome there are three known miR-133 genes: miR-133a-1, miR-133a-2 and miR-133b found on chromosomes 18, 20 and 6 respectively. The mature sequence is excised from the 3′ arm of the hairpin. miR-133 is expressed in muscle tissue and appears to repress the expression of non-muscle genes. [2]

Micro RNA 206 (miR206). MiR-206 is a microRNA that in humans is a member of the myomiR family which also includes miR-1, miR-133, miR-208a/b among few others. [1][2][3][4]

As well as being regulated during the embryonic development of skeletal muscle, miR-206 is regulated by estradiol.[5][6] C2C12 myoblast cells are widely used as a model for the study of cell differentiation in skeletal muscle. Furthermore, miR-206 is highly expressed in triple-negative breast tumors that grow independent of estradiol, and miR-206 is a predictor of worse overall survival in breast cancer patients. [7]

The biogenesis of miR-206 is unique in that the primary mature transcript is generated from the 3p arm of the precursor hairpin rather than the 5p arm. [8] miR-206 has 12 additional family members, whereby the seed sequence is 100% conserved across all miRNAs within the family.

Single nucleotide polymorphisms (SNPs) are also present in the miRNA sequence, some of them with functional consequences, in the sense that the efficiency of miRNA binding to a cognate mRNA target is altered depending on a single nucleotide substitution. DI In fact a number of studies have indicated that the canonical seed sequence of a miRNA is not longer the sole determinate in miRNA:mRNA pairing interactions, as mutations of residues outside the seed region alters binding efficacy.

miR-206 is of interest due to the continued detection of this miRNA in samples from those with type 2 diabetes and non-alcoholic fatty liver disease (NAFLD). In some studies, the therapeutic delivery of miR-206 in a dietary obese mouse model resulted in reduced lipid and glucose production within the liver. The ability of miR-206 to facilitate insulin signaling and modulate lipogenesis indicates miR-206 may be a novel therapy for those with hyperglycemia.[10]

Exosome Isolation Methods. Exosomes are secreted by most cells types and, therefore, may be isolated from cell culture as well as bodily fluids, including plasma, urine, saliva, serum, and cerebrospinal fluid. [3] Protocols for the isolation of exosomes have been tailored for each sample type by varying parameters such as speed of centrifugation, use of filtration and density gradients, and other techniques. [1] The most widely practiced method of isolating and purifying exosomes from various sources is differential ultracentrifugation, [4] in which a series of spins facilitates the stepwise reduction of contaminants, like dead cells, cell debris, platelets, proteins, and nucleic acid complexes and aggregates, and other contaminants from the isolate, [1] however, non-exosomal contaminants, like lipoprotein particles, viruses and bacteria, microsomes, DNA and products of necrosis (such as apoptotic bodies), and protein aggregates, may still remain.

Other techniques for exosome isolation, often used in combination with differential centrifugation, include: iodixanol density gradient, precipitation, ultrafiltration, immunoaffinity isolation, size exclusion chromatography, and microfluidics techniques. [5] Each method has advantages and drawbacks, so attention should be given to selecting a method that best serves the downstream application. In addition, some isolation and purification methods may produce the highest yield of protein but render the vesicles non-viable for certain applications. [5] For example, size exclusion chromatography has been shown to produce highly pure samples with relatively low contamination, cost, and processing time, however, the final sample may be significantly diluted, only one sample may be processed at a time, and the process can be labor intensive. [5]

Non-exosome content within a sample represents a significant challenge for the analysis of exosome characteristics, as well as to their utilization in assays and other approaches. [1] Some contaminants that can be difficult to remove from vesicle preparations include lipoprotein particles, microbes, microsomes, protein aggregates, as well as DNA and other products of necrosis. [1] Depending on the intended downstream application of the purified exosomes, further steps may be taken to remove contaminants from the purified exosome sample. For example, it has been demonstrated that exosome preparations generated with an iodixanol density gradient achieve high purity, as evidenced by enrichment of the exosomal marker CD63 and absence of contaminating proteins, like extracellular Argonaute-2 complexes. [6] A common method for estimating the amount of contaminating protein is by comparing the ratio of nano-vesicle counts (obtained through vesicle visualization technologies such as the NanoSight platform) to protein concentration (obtained with colorimetric protein assays such as the BCA assay). [7] The presence of contaminating proteins alters this ratio and can therefore provide an estimate of the purity of the exosomal sample. It has been reported that samples with ratios greater than 3×1010 particles per μg protein are considered highly pure, while ratios of 2×109 to 2×1010 particles per μg protein are considered of low purity. [7] When developing an isolation protocol, it is recommended that the sample be evaluated and optimized for not only the presence of exosomes, but also for the absence of contaminating factors.

Exosome Characterization. Several common strategies are used to ascertain exosome quantity and purity, and further for further characterization of vesicles following purification. [5] Dynamic light scattering (DLS) analysis, when used in concert with analytical ultracentrifugation, selectively enriches for exosomes within a particular size range, based on their light scattering signatures. Mass spectrometry may be used to identify protein cargo in exosomes, and determine whether the sample may contain protein aggregates or other contaminants. [5] Electron microscopy can be used to visualize vesicle morphology and size, and when used in combination with immuno-labeling, specific features of exosomes, such as surface proteins. [1] Conventional Western blotting may detect the presence of certain proteins in the sample, but cannot determine exosome quantity. Other techniques that have been used to assess the quality and quantity of exosome preparations include: atomic force microscopy, optical single particle tracking, flow cytometry, and resistive pulse sensing. [1]

A number of exosomal markers have been identified and used to confirm the presence of exosomes in preparations.⁸ Common protein markers include Alix, TSG101, CD9, and CD63, and other proteins thought to be enriched in exosomes include DIP2B and members of the 4-transmembrane protein family. [2] However, not all exosomes contain these proteins. The online database ExoCarta (http://www.exocarta.org/) is a tool to help researchers identify and characterize exosomal cargoes. The database contains proteins, RNA sequences, and lipids that have been identified in specific exosomal preparations. Ultimately, identification of protein markers from tumor-derived exosomes, for example, may be used to develop clinical diagnostic testing for tumors in patients.

Standardization of exosome isolation and characterization is an essential step for reliable and reproducible results from assays and other downstream applications, including clinical and therapeutic applications. The myriad sources of exosomal collection, as well as the variety of isolation techniques, has presented many challenges to standardizing protocols and obtaining consistent and reproducible results. [1]

Mapping of Identified miRNA

Analysis of three different stromal cells, one obtained from low passage adult mesenchymal stem cells and two from different low passage fetal cardiac stromal cell lines were characterized for miRNA expression and sequencing. Cells were washed with buffer, trypsinized, and TRIzol® Reagent was used to preserve mRNA.

The expression of known and unique miRNAs in each sample are statistically analyzed and normalized by Transcripts Per Million or TPM [63]. The normalized expression=(read count*1,000,000)/libsize. Libsize is the sample miRNA read count and certain applications may be identified.

In this analysis, the average TPM for the CStr lines was more than 2 fold that of the adult MSC line for the following miRNA: hsa-miR-146a-5p, hsa-miR-490-3p, hsa-miR-9-5p, hsa-miR-3117-3p, hsa-miR-4521, hsa-miR-412-5p, hsa-miR-541-3p, hsa-miR-6724-5p, hsa-miR-182-5p, hsa-miR-454-5p, hsa-miR-206, hsa-miR-584-5p, hsa-miR-7706, hsa-miR-3177-3p, hsa-miR-410-5p, hsa-miR-541-5p, hsa-miR-3175, hsa-miR-204-5p, hsa-miR-3661, hsa-miR-302a-5p, hsa-miR-4661-5p, hsa-miR-543, hsa-miR-103a-2-5p, hsa-miR-3176, hsa-miR-433-5p, hsa-miR-10a-5p, hsa-miR-6753-3p, hsa-miR-330-5p, hsa-miR-11401, hsa-miR-582-3p, hsa-miR-2355-3p, hsa-miR-6511b-5p, hsa-miR-494-5p, hsa-miR-548k, hsa-miR-200a-3p, hsa-miR-744-3p, hsa-miR-487a-3p, hsa-miR-4665-5p, hsa-miR-598-3p, hsa-miR-548ao-3p, hsa-miR-301a-5p, hsa-miR-940, hsa-miR-323a-5p, hsa-miR-1228-5p, hsa-miR-760, hsa-miR-495-3p hsa-miR-937-3p, hsa-miR-4684-3p, hsa-miR-337-3p, hsa-miR-3138, hsa-miR-433-3p, hsa-miR-487b-5p, hsa-miR-2682-5p, hsa-miR-6732-3p, hsa-miR-3167, hsa-miR-3187-3p, hsa-miR-219a-1-3p, hsa-miR-18a-3p, hsa-miR-1343-3p, hsa-miR-98-5p, hsa-miR-191-3p, hsa-miR-33a-3p, hsa-miR-143-3p, hsa-miR-432-5p, hsa-miR-548ay-3p, hsa-miR-1307-3p, hsa-miR-8485, hsa-miR-487a-5p, hsa-miR-451a, hsa-miR-33b-3p, hsa-miR-3155b, hsa-miR-380-5p, hsa-miR-10401-3p, hsa-miR-539-5p hsa-miR-323b-3p, hsa-miR-3605-3p, hsa-miR-3064-5p, hsa-miR-3691-5p, hsa-miR-6827-5p, hsa-miR-487b-3p, hsa-miR-3074-5p, hsa-miR-100-5p, hsa-miR-24-3p, hsa-miR-149-5p, hsa-miR-1909-3p, hsa-miR-3675-3p, hsa-miR-323a-3p, hsa-miR-129-5p, hsa-miR-187-3p, hsa-miR-431-5p, and hsa-miR-200a-5p.

Exosomes with higher fold TPM of two or more miR from fetal derived tissue specific stromal cells than adult mesenchymal stem cells, in this case fetal cardiac stromal cells, are central to the invention here. Fetal cardiac stromal cells here show hsa-miR-206 has a consistent 7.5 fold TPM that that in adult MSC.

hsa-miR-148a-3p has a much higher TPM in MSC than in either CStr cells (see table below). Overexpression of miR-148a/b-3p in ECs has been shown to significantly reduce migration, filamentous actin remodeling, and angiogenic sprouting. It has been identified from endothelial cells as a therapeutic candidate for overcoming EC dysfunction and angiogenic disorders, including ischemia, and retinopathy.

hsa-miR-10a-5p has a much higher TPM in both CStr cell populations than in the MSC population (see table 5 below). This miRNA is associated with negative regulation of cardiomyocyte proliferation. This suggests a therapeutic role of CStr cells and cell derived therapeutics involving this miRNA for the treatment of cardiac diseases where enhanced cardiomyocyte proliferation is desired (such as heart failure, myocardial infarction) and where reduced cardiomyocyte proliferation has potential therapeutic benefits such as cardiac hyperplasia or heart failure of preserved ejection fraction.

Such effects can be synergistic as well as often proliferating cardiomyocytes need new blood vessels to provide for nutrients and waste disposal. Thus CStr cellular and cell derived products such as exosomes containing these and other miRNA as well as specific isolated miRNA have therapeutic potential for these cardiac indications.

TABLE 5
Table of TMP for Three Stromal Cell Types

| sRNA.readcount | MSC | CStr-1 | CStr-4 |
|---|---|---|---|
| hsa-miR-21-5p | 504905 | 117474 | 296995 |
| hsa-miR-100-5p | 235569 | 668104 | 332175 |
| hsa-miR-143-3p | 57194 | 53543 | 229168 |
| hsa-miR-148a-3p | 19073 | 72 | 104 |
| hsa-miR-222-3p | 12078 | 13373 | 5862 |
| hsa-miR-99b-5p | 9768 | 15417 | 10396 |
| hsa-miR-125b-5p | 9636 | 8267 | 3976 |
| hsa-miR-26a-5p | 9602 | 6498 | 4986 |
| hsa-miR-30d-5p | 8407 | 3897 | 3457 |
| hsa-let-7i-5p | 7299 | 2474 | 6652 |
| hsa-miR-145-5p | 6530 | 544 | 2572 |
| hsa-miR-10a-5p | 6397 | 44022 | 10917 |
| hsa-miR-199a-5p | 5575 | 914 | 3184 |
| hsa-miR-27b-3p | 5388 | 997 | 4658 |
| hsa-miR-22-3p | 5191 | 1386 | 2771 |
| hsa-miR-127-3p | 4221 | 4735 | 5989 |
| hsa-let-7a-5p | 4211 | 2640 | 1986 |
| hsa-miR-151a-3p | 4017 | 4556 | 7608 |
| hsa-miR-10b-5p | 3977 | 34 | 26 |
| hsa-miR-30a-5p | 3953 | 2380 | 1337 |
| hsa-let-7b-5p | 3698 | 392 | 238 |
| hsa-miR-140-3p | 3396 | 354 | 962 |
| hsa-miR-221-3p | 2455 | 2436 | 2107 |
| hsa-miR-370-3p | 2025 | 4700 | 2965 |
| hsa-miR-99a-5p | 1950 | 440 | 1433 |
| hsa-miR-423-3p | 1895 | 1742 | 1000 |
| hsa-miR-3184-5p | 1892 | 1740 | 999 |
| hsa-miR-181a-5p | 1864 | 241 | 624 |
| hsa-miR-409-3p | 1732 | 3501 | 2901 |
| hsa-miR-24-3p | 1655 | 2068 | 4959 |
| hsa-miR-3074-5p | 1649 | 2064 | 4947 |
| hsa-miR-199a-3p | 1592 | 155 | 688 |
| hsa-miR-125a-5p | 1483 | 720 | 588 |
| hsa-miR-381-3p | 1462 | 874 | 1783 |
| hsa-miR-191-5p | 1435 | 1378 | 836 |
| hsa-miR-92a-3p | 1406 | 1574 | 1323 |
| hsa-miR-214-3p | 1247 | 136 | 387 |
| hsa-let-7f-5p | 1185 | 1136 | 1824 |
| hsa-miR-320a-3p | 1157 | 408 | 460 |
| hsa-miR-3184-3p | 992 | 362 | 412 |
| hsa-miR-423-5p | 992 | 362 | 412 |
| hsa-miR-221-5p | 947 | 342 | 539 |
| hsa-miR-103a-3p | 931 | 811 | 1460 |
| hsa-miR-125b-1-3p | 906 | 439 | 394 |
| hsa-miR-23a-3p | 901 | 545 | 1218 |
| hsa-miR-103b | 887 | 783 | 1389 |
| hsa-miR-29a-3p | 863 | 598 | 496 |
| hsa-miR-193b-3p | 857 | 129 | 268 |
| hsa-miR-361-3p | 838 | 245 | 427 |
| hsa-let-7g-5p | 816 | 840 | 1068 |
| hsa-miR-30c-5p | 816 | 454 | 277 |
| hsa-let-7e-5p | 769 | 351 | 722 |
| hsa-miR-615-3p | 756 | 195 | 3 |
| hsa-miR-224-5p | 694 | 69 | 134 |
| hsa-miR-411-5p | 646 | 287 | 564 |
| hsa-miR-379-5p | 629 | 417 | 877 |
| hsa-miR-152-3p | 600 | 205 | 532 |
| hsa-miR-146b-5p | 599 | 21 | 22 |
| hsa-miR-28-3p | 546 | 308 | 534 |
| hsa-miR-574-3p | 524 | 187 | 327 |
| hsa-miR-193a-5p | 514 | 81 | 118 |
| hsa-miR-30e-5p | 497 | 91 | 123 |
| hsa-miR-92b-3p | 496 | 284 | 209 |
| hsa-miR-23b-3p | 492 | 164 | 628 |
| hsa-miR-574-5p | 489 | 194 | 228 |
| hsa-miR-27a-3p | 484 | 215 | 514 |
| hsa-miR-1307-3p | 464 | 1164 | 1035 |
| hsa-miR-335-3p | 463 | 106 | 22 |
| hsa-miR-654-3p | 450 | 659 | 775 |
| hsa-miR-7-5p | 448 | 303 | 401 |
| hsa-miR-3529-3p | 441 | 300 | 399 |
| hsa-miR-374b-5p | 411 | 38 | 123 |
| hsa-miR-374c-3p | 411 | 38 | 123 |
| hsa-miR-941 | 408 | 261 | 312 |
| hsa-miR-181a-3p | 394 | 97 | 334 |
| hsa-miR-181a-2-3p | 387 | 310 | 567 |
| hsa-miR-365a-3p | 383 | 49 | 138 |
| hsa-miR-452-5p | 348 | 10 | 38 |
| hsa-miR-744-5p | 339 | 461 | 368 |
| hsa-miR-532-5p | 335 | 88 | 278 |
| hsa-miR-210-3p | 324 | 13 | 47 |
| hsa-miR-425-5p | 319 | 103 | 169 |
| hsa-miR-155-5p | 316 | 266 | 387 |
| hsa-miR-21-3p | 306 | 21 | 101 |
| hsa-miR-31-5p | 300 | 61 | 179 |
| hsa-miR-17-5p | 300 | 36 | 72 |
| hsa-miR-99b-3p | 289 | 291 | 476 |
| hsa-miR-27a-5p | 288 | 207 | 504 |
| hsa-miR-106b-3p | 285 | 128 | 146 |
| hsa-miR-101-3p | 275 | 16 | 78 |
| hsa-miR-493-3p | 262 | 455 | 211 |
| hsa-miR-424-3p | 257 | 77 | 143 |
| hsa-miR-197-3p | 255 | 148 | 188 |
| hsa-miR-148b-3p | 255 | 110 | 254 |
| hsa-miR-340-5p | 250 | 7 | 23 |
| hsa-miR-134-5p | 237 | 273 | 316 |
| hsa-miR-199b-5p | 232 | 107 | 400 |
| hsa-miR-93-5p | 232 | 168 | 173 |
| hsa-miR-16-5p | 222 | 49 | 99 |
| hsa-miR-708-3p | 216 | 4 | 61 |
| hsa-miR-654-5p | 210 | 367 | 360 |
| hsa-miR-484 | 210 | 103 | 105 |
| hsa-miR-25-3p | 210 | 256 | 256 |
| hsa-miR-708-5p | 206 | 6 | 46 |
| hsa-miR-181b-5p | 199 | 98 | 206 |
| hsa-miR-758-3p | 196 | 115 | 165 |
| hsa-miR-128-3p | 195 | 221 | 223 |
| hsa-miR-378a-3p | 186 | 45 | 334 |
| hsa-miR-339-5p | 175 | 60 | 56 |
| hsa-miR-382-5p | 169 | 183 | 241 |
| hsa-miR-127-5p | 166 | 41 | 87 |
| hsa-miR-409-5p | 165 | 110 | 197 |
| hsa-let-7c-5p | 163 | 19 | 79 |
| hsa-miR-1260b | 158 | 61 | 91 |
| hsa-miR-365a-5p | 156 | 19 | 32 |
| hsa-miR-769-5p | 143 | 65 | 164 |
| hsa-miR-218-5p | 141 | 39 | 38 |
| hsa-miR-494-3p | 138 | 61 | 101 |
| hsa-miR-455-3p | 138 | 23 | 102 |
| hsa-miR-323a-3p | 134 | 270 | 283 |
| hsa-miR-148a-5p | 133 | 0 | 0 |
| hsa-miR-342-3p | 125 | 28 | 43 |
| hsa-miR-132-5p | 118 | 7 | 12 |
| hsa-miR-125b-2-3p | 117 | 8 | 53 |
| hsa-miR-20a-5p | 112 | 22 | 55 |
| hsa-miR-629-5p | 112 | 29 | 65 |
| hsa-miR-4326 | 106 | 101 | 66 |
| hsa-miR-26b-5p | 104 | 45 | 85 |
| hsa-miR-455-5p | 104 | 13 | 75 |
| hsa-miR-132-3p | 102 | 16 | 20 |
| hsa-miR-214-5p | 101 | 8 | 37 |
| hsa-miR-3120-3p | 101 | 8 | 37 |
| hsa-miR-30c-2-3p | 99 | 53 | 27 |
| hsa-miR-22-5p | 98 | 30 | 66 |
| hsa-miR-1180-3p | 94 | 95 | 67 |
| hsa-miR-143-5p | 94 | 17 | 95 |
| hsa-miR-542-3p | 91 | 3 | 6 |
| hsa-miR-136-3p | 91 | 14 | 50 |
| hsa-miR-495-3p | 88 | 173 | 378 |
| hsa-miR-185-5p | 87 | 31 | 61 |
| hsa-miR-369-5p | 86 | 69 | 125 |
| hsa-miR-7974 | 84 | 39 | 85 |
| hsa-miR-193b-5p | 84 | 8 | 17 |
| hsa-miR-145-3p | 83 | 13 | 75 |
| hsa-let-7a-2-3p | 82 | 38 | 32 |
| hsa-let-7b-3p | 82 | 5 | 6 |
| hsa-miR-485-5p | 82 | 79 | 74 |
| hsa-miR-30a-3p | 82 | 202 | 114 |
| hsa-miR-410-3p | 8 | 51 | 104 |
| hsa-miR-3120-5p | 79 | 8 | 22 |
| hsa-miR-664a-3p | 78 | 5 | 24 |
| hsa-miR-411-3p | 77 | 91 | 150 |
| hsa-miR-1-3p | 77 | 59 | 73 |
| hsa-miR-500a-3p | 77 | 11 | 37 |
| hsa-let-7d-5p | 77 | 67 | 71 |
| hsa-miR-3158-3p | 76 | 8 | 22 |
| hsa-miR-589-5p | 74 | 35 | 79 |
| hsa-miR-3158-5p | 74 | 8 | 22 |
| hsa-miR-493-5p | 73 | 78 | 33 |
| hsa-let-7d-3p | 72 | 64 | 69 |
| hsa-miR-149-5p | 71 | 218 | 81 |
| hsa-miR-671-3p | 71 | 50 | 35 |
| hsa-miR-186-5p | 70 | 21 | 71 |
| hsa-miR-1185-1-3p | 69 | 42 | 67 |
| hsa-miR-543 | 69 | 411 | 254 |
| hsa-miR-323b-3p | 68 | 151 | 155 |
| hsa-miR-34a-5p | 67 | 11 | 43 |
| hsa-miR-361-5p | 67 | 22 | 57 |
| hsa-miR-15b-5p | 65 | 46 | 55 |
| hsa-miR-27b-5p | 64 | 19 | 76 |
| hsa-miR-30b-5p | 64 | 11 | 27 |
| hsa-miR-339-3p | 62 | 20 | 28 |
| hsa-miR-328-3p | 61 | 37 | 18 |
| hsa-miR-24-2-5p | 59 | 19 | 51 |
| hsa-miR-28-5p | 58 | 40 | 7 |
| hsa-miR-365b-5p | 56 | 15 | 13 |
| hsa-miR-19b-3p | 54 | 2 | 8 |
| hsa-miR-3615 | 53 | 61 | 53 |
| hsa-miR-1271-5p | 51 | 29 | 12 |
| hsa-miR-1296-5p | 50 | 19 | 19 |
| hsa-miR-130b-5p | 47 | 74 | 36 |
| hsa-miR-485-3p | 46 | 46 | 55 |
| hsa-miR-4485-3p | 45 | 1 | 6 |
| hsa-miR-625-3p | 45 | 5 | 12 |
| hsa-miR-125a-3p | 45 | 9 | 20 |
| hsa-miR-210-5p | 44 | 4 | 10 |
| hsa-miR-140-5p | 42 | 4 | 13 |
| hsa-miR-502-3p | 42 | 5 | 21 |
| hsa-miR-345-5p | 40 | 22 | 12 |
| hsa-miR-146b-3p | 37 | 1 | 1 |
| hsa-miR-330-3p | 37 | 51 | 50 |
| hsa-miR-374a-5p | 36 | 2 | 7 |
| hsa-let-7e-3p | 35 | 12 | 20 |
| hsa-miR-195-3p | 35 | 3 | 1 |
| hsa-miR-2682-5p | 34 | 88 | 102 |
| hsa-miR-335-5p | 34 | 2 | 0 |
| hsa-miR-10395-3p | 33 | 3 | 13 |
| hsa-miR-503-5p | 32 | 27 | 64 |
| hsa-miR-433-3p | 32 | 111 | 70 |
| hsa-miR-549a-5p | 31 | 5 | 3 |
| hsa-miR-382-3p | 30 | 20 | 61 |
| hsa-miR-889-3p | 30 | 19 | 51 |
| hsa-miR-126-3p | 30 | 14 | 87 |
| hsa-miR-3909 | 29 | 14 | 14 |
| hsa-miR-549a-3p | 29 | 5 | 3 |
| hsa-miR-432-5p | 27 | 52 | 80 |
| hsa-miR-151a-5p | 27 | 15 | 30 |
| hsa-let-7i-3p | 27 | 2 | 6 |
| hsa-miR-30e-3p | 27 | 32 | 63 |
| hsa-miR-107 | 26 | 18 | 27 |
| hsa-miR-296-3p | 26 | 14 | 13 |
| hsa-miR-501-3p | 26 | 9 | 23 |
| hsa-miR-192-5p | 26 | 11 | 25 |
| hsa-miR-138-5p | 25 | 1 | 6 |
| hsa-miR-320b | 25 | 9 | 17 |
| hsa-miR-34c-5p | 25 | 9 | 14 |
| hsa-miR-4286 | 25 | 13 | 11 |
| hsa-miR-377-5p | 25 | 13 | 12 |
| hsa-miR-329-3p | 24 | 20 | 17 |
| hsa-miR-10a-3p | 24 | 32 | 26 |
| hsa-miR-129-5p | 24 | 22 | 76 |
| hsa-miR-431-5p | 24 | 36 | 59 |
| hsa-miR-450a-5p | 23 | 7 | 13 |
| hsa-miR-500a-5p | 22 | 4 | 11 |
| hsa-miR-196b-5p | 22 | 1 | 0 |
| hsa-miR-874-3p | 21 | 8 | 5 |
| hsa-miR-31-3p | 21 | 1 | 15 |
| hsa-miR-331-3p | 21 | 3 | 8 |
| hsa-miR-299-5p | 20 | 7 | 14 |
| hsa-miR-625-5p | 20 | 3 | 8 |
| hsa-miR-362-5p | 19 | 3 | 9 |
| hsa-miR-585-3p | 19 | 0 | 0 |
| hsa-miR-1268a | 19 | 10 | 24 |
| hsa-miR-548o-3p | 19 | 9 | 24 |
| hsa-miR-424-5p | 17 | 3 | 16 |
| hsa-miR-379-3p | 17 | 8 | 18 |
| hsa-miR-450b-5p | 17 | 3 | 7 |
| hsa-miR-505-3p | 16 | 10 | 15 |
| hsa-miR-196a-5p | 16 | 0 | 0 |
| hsa-miR-299-3p | 16 | 3 | 4 |
| hsa-miR-331-5p | 16 | 5 | 6 |
| hsa-miR-195-5p | 15 | 0 | 0 |
| hsa-miR-152-5p | 15 | 6 | 16 |
| hsa-miR-425-3p | 14 | 17 | 15 |
| hsa-miR-576-3p | 14 | 1 | 6 |
| hsa-miR-181c-3p | 14 | 6 | 8 |
| hsa-miR-877-5p | 14 | 20 | 33 |
| hsa-miR-106b-5p | 14 | 4 | 7 |
| hsa-miR-1185-2-3p | 14 | 7 | 9 |
| hsa-miR-660-5p | 14 | 2 | 10 |
| hsa-miR-532-3p | 13 | 2 | 5 |
| hsa-miR-224-3p | 13 | 0 | 1 |
| hsa-miR-193a-3p | 12 | 0 | 1 |
| hsa-miR-92b-5p | 12 | 9 | 5 |
| hsa-let-7a-3p | 12 | 2 | 1 |
| hsa-miR-130a-3p | 12 | 2 | 4 |
| hsa-miR-1303 | 12 | 9 | 6 |
| hsa-miR-659-5p | 12 | 1 | 5 |
| hsa-miR-377-3p | 11 | 0 | 2 |
| hsa-miR-618 | 11 | 2 | 0 |
| hsa-miR-431-3p | 11 | 11 | 20 |
| hsa-miR-3940-3p | 11 | 1 | 1 |
| hsa-miR-539-3p | 10 | 1 | 3 |
| hsa-miR-181d-5p | 10 | 6 | 5 |
| hsa-miR-222-5p | 10 | 11 | 14 |
| hsa-miR-376c-3p | 10 | 1 | 8 |
| hsa-miR-487b-3p | 10 | 20 | 23 |
| hsa-miR-340-3p | 10 | 4 | 6 |
| hsa-miR-4454 | 9 | 0 | 0 |
| hsa-miR-370-5p | 9 | 14 | 12 |
| hsa-miR-374a-3p | 9 | 0 | 1 |
| hsa-miR-454-3p | 9 | 2 | 5 |
| hsa-miR-7706 | 9 | 69 | 70 |
| hsa-miR-148b-5p | 9 | 2 | 8 |
| hsa-miR-30c-1-3p | 9 | 2 | 4 |
| hsa-miR-671-5p | 9 | 2 | 2 |
| hsa-miR-98-5p | 9 | 17 | 28 |
| hsa-miR-652-3p | 9 | 8 | 12 |
| hsa-miR-483-3p | 9 | 2 | 0 |
| hsa-miR-497-5p | 9 | 0 | 0 |
| hsa-miR-10b-3p | 8 | 0 | 0 |
| hsa-miR-369-3p | 8 | 3 | 9 |
| hsa-miR-4677-3p | 8 | 1 | 4 |
| hsa-miR-16-2-3p | 8 | 14 | 9 |
| hsa-miR-18a-5p | 8 | 2 | 4 |
| hsa-miR-337-3p | 8 | 16 | 32 |
| hsa-miR-641 | 8 | 2 | 4 |
| hsa-miR-486-3p | 8 | 20 | 4 |
| hsa-miR-486-5p | 8 | 20 | 4 |
| hsa-miR-10401-3p | 7 | 15 | 18 |
| hsa-miR-1301-3p | 7 | 8 | 5 |
| hsa-miR-15a-5p | 7 | 1 | 2 |
| novel_10 | 7 | 6 | 19 |
| hsa-miR-1287-5p | 7 | 11 | 8 |
| hsa-miR-656-3p | 7 | 3 | 9 |
| hsa-miR-615-5p | 7 | 0 | 0 |
| hsa-miR-1285-3p | 7 | 2 | 3 |
| hsa-miR-3200-3p | 7 | 4 | 4 |
| hsa-miR-185-3p | 6 | 5 | 4 |
| hsa-miR-93-3p | 6 | 3 | 2 |
| hsa-miR-15b-3p | 6 | 6 | 10 |
| hsa-miR-133a-3p | 6 | 6 | 9 |
| hsa-miR-576-5p | 6 | 2 | 8 |
| hsa-miR-766-3p | 6 | 4 | 9 |
| hsa-miR-29a-5p | 5 | 1 | 2 |
| hsa-miR-200b-3p | 5 | 2 | 7 |
| hsa-miR-23a-5p | 5 | 2 | 4 |
| hsa-miR-29b-3p | 5 | 1 | 1 |
| hsa-miR-496 | 5 | 6 | 10 |
| hsa-miR-1292-5p | 5 | 6 | 7 |
| hsa-miR-130b-3p | 5 | 1 | 2 |
| hsa-miR-1843 | 5 | 3 | 9 |
| hsa-miR-194-5p | 5 | 0 | 1 |
| hsa-miR-378c | 5 | 0 | 6 |
| hsa-miR-548e-3p | 5 | 0 | 2 |
| hsa-miR-550a-3-5p | 5 | 2 | 2 |
| hsa-miR-550a-5p | 5 | 2 | 2 |
| hsa-miR-128-1-5p | 5 | 5 | 6 |
| hsa-miR-5701 | 5 | 0 | 0 |
| hsa-miR-2110 | 4 | 3 | 2 |
| hsa-miR-217-5p | 4 | 1 | 9 |
| hsa-miR-4746-5p | 4 | 3 | 3 |
| hsa-miR-25-5p | 4 | 6 | 3 |
| hsa-miR-12136 | 4 | 1 | 1 |
| hsa-miR-4521 | 4 | 91 | 80 |
| hsa-miR-887-3p | 4 | 1 | 2 |
| hsa-miR-139-5p | 4 | 3 | 6 |
| hsa-miR-30b-3p | 4 | 1 | 0 |
| hsa-miR-483-5p | 4 | 1 | 0 |
| hsa-miR-146a-5p | 4 | 6 | 1639 |
| hsa-miR-23b-5p | 4 | 2 | 6 |
| hsa-miR-29c-5p | 4 | 3 | 3 |
| hsa-miR-342-5p | 4 | 2 | 2 |
| hsa-miR-376a-3p | 4 | 2 | 8 |
| hsa-miR-655-3p | 4 | 1 | 3 |
| hsa-miR-1278 | 4 | 0 | 3 |
| hsa-miR-137-3p | 4 | 1 | 3 |
| hsa-miR-3152-5p | 4 | 1 | 3 |
| hsa-miR-378a-5p | 4 | 1 | 7 |
| hsa-miR-4473 | 4 | 2 | 1 |
| hsa-miR-501-5p | 4 | 2 | 5 |
| hsa-miR-584-5p | 4 | 31 | 23 |
| hsa-miR-92a-1-5p | 4 | 5 | 4 |
| hsa-miR-1248 | 3 | 1 | 1 |
| hsa-miR-17-3p | 3 | 1 | 2 |
| hsa-miR-206 | 3 | 26 | 26 |
| hsa-miR-542-5p | 3 | 0 | 1 |
| hsa-miR-664b-3p | 3 | 1 | 2 |
| hsa-miR-1306-5p | 3 | 3 | 3 |
| hsa-miR-154-5p | 3 | 2 | 5 |
| hsa-miR-19a-3p | 3 | 0 | 0 |
| hsa-miR-2277-5p | 3 | 2 | 1 |
| hsa-miR-629-3p | 3 | 1 | 0 |
| hsa-miR-7704 | 3 | 1 | 0 |
| hsa-miR-1255a | 3 | 0 | 1 |
| hsa-miR-130a-5p | 3 | 2 | 2 |
| hsa-miR-135b-5p | 3 | 0 | 0 |
| hsa-miR-665 | 3 | 1 | 2 |
| hsa-miR-7-1-3p | 3 | 1 | 2 |
| hsa-miR-1197 | 3 | 3 | 5 |
| hsa-miR-1245a | 3 | 0 | 2 |
| hsa-miR-1294 | 3 | 1 | 2 |
| hsa-miR-138-1-3p | 3 | 0 | 1 |
| hsa-miR-3651 | 3 | 0 | 1 |
| hsa-miR-421 | 3 | 1 | 5 |
| hsa-miR-539-5p | 3 | 4 | 9 |
| hsa-miR-1293 | 3 | 1 | 6 |
| hsa-miR-3129-3p | 3 | 1 | 1 |
| hsa-miR-3934-5p | 3 | 2 | 1 |
| hsa-miR-154-3p | 3 | 0 | 1 |
| hsa-miR-200c-3p | 3 | 2 | 4 |
| hsa-miR-212-5p | 3 | 3 | 2 |
| hsa-miR-30d-3p | 3 | 1 | 1 |
| hsa-miR-34c-3p | 3 | 2 | 1 |
| hsa-miR-487a-5p | 3 | 6 | 6 |
| hsa-miR-585-5p | 3 | 0 | 0 |
| hsa-miR-6515-5p | 3 | 0 | 1 |
| hsa-miR-663b | 3 | 0 | 0 |
| hsa-miR-668-3p | 3 | 2 | 1 |
| hsa-let-7c-3p | 2 | 0 | 2 |
| hsa-miR-2116-3p | 2 | 0 | 1 |
| hsa-miR-32-5p | 2 | 0 | 0 |
| hsa-miR-500b-5p | 2 | 0 | 0 |
| hsa-miR-129-2-3p | 2 | 1 | 3 |
| hsa-miR-320c | 2 | 1 | 2 |
| hsa-miR-500b-3p | 2 | 1 | 1 |
| hsa-miR-548u | 2 | 0 | 0 |
| hsa-miR-6715a-3p | 2 | 0 | 0 |
| hsa-miR-1307-5p | 2 | 1 | 1 |
| hsa-miR-136-5p | 2 | 0 | 1 |
| hsa-miR-337-5p | 2 | 1 | 2 |
| hsa-miR-6715b-5p | 2 | 0 | 0 |
| hsa-miR-6852-5p | 2 | 1 | 1 |
| novel_5 | 2 | 0 | 0 |
| hsa-miR-100-3p | 2 | 0 | 0 |
| hsa-miR-1304-5p | 2 | 1 | 2 |
| hsa-miR-34b-3p | 2 | 2 | 1 |
| hsa-miR-378d | 2 | 0 | 2 |
| hsa-miR-450a-2-3p | 2 | 1 | 1 |
| hsa-miR-548p | 2 | 1 | 2 |
| hsa-miR-6715b-3p | 2 | 0 | 0 |
| novel_2 | 2 | 2 | 8 |
| hsa-miR-24-1-5p | 2 | 1 | 1 |
| hsa-miR-5010-3p | 2 | 1 | 1 |
| hsa-miR-1245b-3p | 2 | 0 | 3 |
| hsa-miR-181b-2-3p | 2 | 0 | 1 |
| hsa-miR-216a-3p | 2 | 0 | 5 |
| hsa-miR-3133 | 2 | 0 | 0 |
| hsa-miR-324-5p | 2 | 1 | 1 |
| hsa-miR-3622a-5p | 2 | 3 | 0 |
| hsa-miR-3622b-3p | 2 | 3 | 0 |
| hsa-miR-374b-3p | 2 | 0 | 1 |
| hsa-miR-380-5p | 2 | 3 | 4 |
| hsa-miR-412-5p | 2 | 4 | 38 |
| hsa-miR-548bc | 2 | 0 | 0 |
| hsa-miR-548f-5p | 2 | 0 | 0 |
| novel_6 | 2 | 0 | 1 |
| hsa-miR-1291 | 1 | 1 | 0 |
| hsa-miR-16-1-3p | 1 | 0 | 0 |
| hsa-miR-181b-3p | 1 | 0 | 1 |
| hsa-miR-219a-1-3p | 1 | 3 | 5 |
| hsa-miR-3074-3p | 1 | 0 | 1 |
| hsa-miR-3173-5p | 1 | 0 | 0 |
| hsa-miR-326 | 1 | 0 | 1 |
| hsa-miR-3617-5p | 1 | 0 | 0 |
| hsa-miR-4781-3p | 1 | 0 | 1 |
| hsa-miR-548f-3p | 1 | 0 | 0 |
| hsa-miR-6842-3p | 1 | 1 | 4 |
| hsa-miR-769-3p | 1 | 1 | 1 |
| hsa-miR-95-3p | 1 | 1 | 0 |
| novel_8 | 1 | 0 | 2 |
| hsa-miR-1228-3p | 1 | 1 | 1 |
| hsa-miR-1468-5p | 1 | 1 | 1 |
| hsa-miR-18a-3p | 1 | 4 | 3 |
| hsa-miR-191-3p | 1 | 4 | 3 |
| hsa-miR-3179 | 1 | 0 | 0 |
| hsa-miR-324-3p | 1 | 2 | 2 |
| hsa-miR-330-5p | 1 | 3 | 7 |
| hsa-miR-376a-5p | 1 | 0 | 1 |
| hsa-miR-487a-3p | 1 | 3 | 6 |
| hsa-miR-499a-5p | 1 | 1 | 2 |
| hsa-miR-548ah-3p | 1 | 1 | 1 |
| hsa-miR-550b-3p | 1 | 0 | 1 |
| hsa-miR-616-5p | 1 | 0 | 1 |
| hsa-miR-6511b-3p | 1 | 1 | 1 |
| hsa-miR-651-5p | 1 | 0 | 0 |
| hsa-miR-664a-5p | 1 | 0 | 2 |
| hsa-miR-6866-5p | 1 | 0 | 2 |
| hsa-miR-9903 | 1 | 0 | 0 |
| hsa-miR-10527-5p | 1 | 0 | 1 |
| hsa-miR-106a-5p | 1 | 0 | 0 |
| hsa-miR-1245b-5p | 1 | 0 | 1 |
| hsa-miR-1284 | 1 | 0 | 0 |
| hsa-miR-2355-5p | 1 | 0 | 0 |
| hsa-miR-29b-1-5p | 1 | 2 | 1 |
| hsa-miR-3174 | 1 | 0 | 0 |
| hsa-miR-3180 | 1 | 1 | 1 |
| hsa-miR-3180-3p | 1 | 1 | 1 |
| hsa-miR-320d | 1 | 0 | 1 |
| hsa-miR-3912-3p | 1 | 0 | 0 |
| hsa-miR-451a | 1 | 2 | 3 |
| hsa-miR-4683 | 1 | 0 | 0 |
| hsa-miR-4787-3p | 1 | 0 | 0 |
| hsa-miR-491-5p | 1 | 0 | 1 |
| hsa-miR-582-3p | 1 | 0 | 9 |
| hsa-miR-597-3p | 1 | 1 | 1 |
| hsa-miR-619-5p | 1 | 1 | 2 |
| hsa-miR-6716-3p | 1 | 2 | 2 |
| hsa-miR-6877-5p | 1 | 1 | 0 |
| hsa-miR-7976 | 1 | 1 | 2 |
| hsa-miR-887-5p | 1 | 1 | 0 |
| hsa-let-7f-1-3p | 1 | 0 | 1 |
| hsa-let-7g-3p | 1 | 0 | 0 |
| hsa-miR-10392-5p | 1 | 1 | 0 |
| hsa-miR-129-1-3p | 1 | 0 | 0 |
| hsa-miR-181c-5p | 1 | 1 | 1 |
| hsa-miR-26a-2-3p | 1 | 0 | 1 |
| hsa-miR-26b-3p | 1 | 1 | 1 |
| hsa-miR-33b-3p | 1 | 3 | 1 |
| hsa-miR-3679-5p | 1 | 0 | 0 |
| hsa-miR-3928-3p | 1 | 1 | 0 |
| hsa-miR-3944-3p | 1 | 1 | 0 |
| hsa-miR-4728-5p | 1 | 0 | 0 |
| hsa-miR-503-3p | 1 | 1 | 2 |
| hsa-miR-548j-5p | 1 | 0 | 1 |
| hsa-miR-548w | 1 | 1 | 1 |
| hsa-miR-5690 | 1 | 0 | 1 |
| hsa-miR-605-5p | 1 | 1 | 1 |
| hsa-miR-6720-3p | 1 | 2 | 1 |
| novel_3 | 1 | 0 | 0 |
| hsa-miR-1246 | 1 | 0 | 0 |
| hsa-miR-1249-5p | 1 | 0 | 0 |
| hsa-miR-1273c | 1 | 0 | 0 |
| hsa-miR-1277-5p | 1 | 0 | 0 |
| hsa-miR-1299 | 1 | 1 | 0 |
| hsa-miR-1343-3p | 1 | 4 | 0 |
| hsa-miR-212-3p | 1 | 0 | 1 |
| hsa-miR-323a-5p | 1 | 2 | 3 |
| hsa-miR-32-3p | 1 | 0 | 0 |
| hsa-miR-34a-3p | 1 | 0 | 0 |
| hsa-miR-3605-5p | 1 | 1 | 1 |
| hsa-miR-3662 | 1 | 0 | 1 |
| hsa-miR-3919 | 1 | 0 | 0 |
| hsa-miR-4725-5p | 1 | 0 | 0 |
| hsa-miR-4788 | 1 | 0 | 0 |
| hsa-miR-499b-3p | 1 | 1 | 2 |
| hsa-miR-548at-3p | 1 | 0 | 0 |
| hsa-miR-548h-5p | 1 | 0 | 1 |
| hsa-miR-579-5p | 1 | 1 | 0 |
| hsa-miR-6505-5p | 1 | 0 | 0 |
| hsa-miR-6511a-3p | 1 | 1 | 0 |
| hsa-miR-6734-5p | 1 | 0 | 0 |
| hsa-miR-6770-3p | 1 | 0 | 0 |
| hsa-miR-6772-3p | 1 | 1 | 0 |
| hsa-miR-6797-5p | 1 | 0 | 0 |
| hsa-miR-6811-5p | 1 | 0 | 0 |
| novel_1 | 1 | 1 | 0 |
| novel_14 | 1 | 0 | 0 |
| hsa-miR-1226-3p | 1 | 0 | 1 |
| hsa-miR-1262 | 1 | 0 | 1 |
| hsa-miR-1285-5p | 1 | 1 | 0 |
| hsa-miR-147b-3p | 1 | 0 | 0 |
| hsa-miR-184 | 1 | 0 | 1 |
| hsa-miR-187-3p | 1 | 0 | 3 |
| hsa-miR-1908-5p | 1 | 1 | 1 |
| hsa-miR-219a-2-3p | 1 | 0 | 1 |
| hsa-miR-219b-5p | 1 | 0 | 1 |
| hsa-miR-2355-3p | 1 | 1 | 4 |
| hsa-miR-29b-2-5p | 1 | 0 | 1 |
| hsa-miR-3065-5p | 1 | 0 | 0 |
| hsa-miR-3180-5p | 1 | 0 | 0 |
| hsa-miR-3187-3p | 1 | 3 | 1 |
| hsa-miR-338-3p | 1 | 0 | 0 |
| hsa-miR-3688-3p | 1 | 0 | 0 |
| hsa-miR-374c-5p | 1 | 0 | 0 |
| hsa-miR-380-3p | 1 | 0 | 2 |
| hsa-miR-3913-3p | 1 | 0 | 0 |
| hsa-miR-3913-5p | 1 | 0 | 0 |
| hsa-miR-3916 | 1 | 0 | 0 |
| hsa-miR-432-3p | 1 | 1 | 1 |
| hsa-miR-4659a-3p | 1 | 0 | 0 |
| hsa-miR-4659b-5p | 1 | 0 | 0 |
| hsa-miR-4725-3p | 1 | 1 | 0 |
| hsa-miR-4797-3p | 1 | 0 | 0 |
| hsa-miR-5579-3p | 1 | 0 | 0 |
| hsa-miR-5581-3p | 1 | 0 | 0 |
| hsa-miR-580-3p | 1 | 0 | 0 |
| hsa-miR-610 | 1 | 0 | 0 |
| hsa-miR-628-5p | 1 | 0 | 0 |
| hsa-miR-636 | 1 | 0 | 1 |
| hsa-miR-660-3p | 1 | 0 | 0 |
| hsa-miR-6827-5p | 1 | 2 | 1 |
| hsa-miR-744-3p | 1 | 2 | 3 |
| hsa-miR-874-5p | 1 | 0 | 1 |
| hsa-miR-937-3p | 1 | 3 | 1 |
| hsa-miR-99a-3p | 1 | 0 | 0 |
| hsa-miR-1193 | 0 | 0 | 0 |
| hsa-miR-1271-3p | 0 | 0 | 0 |
| hsa-miR-1305 | 0 | 0 | 0 |
| hsa-miR-1306-3p | 0 | 1 | 0 |
| hsa-miR-134-3p | 0 | 1 | 1 |
| hsa-miR-155-3p | 0 | 0 | 0 |
| hsa-miR-196a-3p | 0 | 0 | 0 |
| hsa-miR-1972 | 0 | 0 | 0 |
| hsa-miR-19b-1-5p | 0 | 0 | 0 |
| hsa-miR-204-5p | 0 | 3 | 3 |
| hsa-miR-216b-5p | 0 | 0 | 0 |
| hsa-miR-223-3p | 0 | 0 | 0 |
| hsa-miR-2277-3p | 0 | 0 | 0 |
| hsa-miR-3117-3p | 0 | 17 | 4 |
| hsa-miR-3121-3p | 0 | 0 | 0 |
| hsa-miR-3125 | 0 | 0 | 0 |
| hsa-miR-3129-5p | 0 | 0 | 0 |
| hsa-miR-3130-3p | 0 | 0 | 0 |
| hsa-miR-3130-5p | 0 | 0 | 0 |
| hsa-miR-3199 | 0 | 0 | 1 |
| hsa-miR-3622a-3p | 0 | 0 | 0 |
| hsa-miR-3648 | 0 | 0 | 0 |
| hsa-miR-3677-3p | 0 | 0 | 0 |
| hsa-miR-3911 | 0 | 0 | 1 |
| hsa-miR-4421 | 0 | 1 | 0 |
| hsa-miR-4449 | 0 | 0 | 0 |
| hsa-miR-449c-5p | 0 | 0 | 0 |
| hsa-miR-4687-5p | 0 | 0 | 0 |
| hsa-miR-4755-3p | 0 | 0 | 0 |
| hsa-miR-4755-5p | 0 | 0 | 0 |
| hsa-miR-4773 | 0 | 0 | 0 |
| hsa-miR-487b-5p | 0 | 2 | 1 |
| hsa-miR-490-3p | 0 | 181 | 0 |
| hsa-miR-491-3p | 0 | 0 | 0 |
| hsa-miR-548aq-3p | 0 | 0 | 0 |
| hsa-miR-548g-5p | 0 | 0 | 0 |
| hsa-miR-5579-5p | 0 | 0 | 0 |
| hsa-miR-5698 | 0 | 0 | 0 |
| hsa-miR-598-3p | 0 | 0 | 3 |
| hsa-miR-6500-3p | 0 | 1 | 0 |
| hsa-miR-6516-3p | 0 | 0 | 0 |
| hsa-miR-6779-5p | 0 | 0 | 0 |
| hsa-miR-6818-3p | 0 | 0 | 0 |
| hsa-miR-6854-5p | 0 | 0 | 0 |
| hsa-miR-6891-5p | 0 | 0 | 0 |
| hsa-miR-939-5p | 0 | 0 | 0 |
| hsa-miR-940 | 0 | 1 | 2 |
| hsa-miR-942-3p | 0 | 0 | 0 |
| hsa-miR-942-5p | 0 | 0 | 0 |
| hsa-miR-9-5p | 0 | 81 | 97 |
| novel_9 | 0 | 0 | 1 |
| hsa-miR-105-5p | 0 | 0 | 0 |
| hsa-miR-11399 | 0 | 0 | 0 |
| hsa-miR-1185-5p | 0 | 0 | 0 |
| hsa-miR-122b-5p | 0 | 0 | 0 |
| hsa-miR-1255b-5p | 0 | 0 | 0 |
| hsa-miR-1260a | 0 | 0 | 0 |
| hsa-miR-1276 | 0 | 0 | 1 |
| hsa-miR-1288-3p | 0 | 1 | 0 |
| hsa-miR-137-5p | 0 | 1 | 1 |
| hsa-miR-139-3p | 0 | 0 | 0 |
| hsa-miR-142-3p | 0 | 0 | 0 |
| hsa-miR-142-5p | 0 | 0 | 0 |
| hsa-miR-147b-5p | 0 | 0 | 0 |
| hsa-miR-182-5p | 0 | 2 | 3 |
| hsa-miR-190a-5p | 0 | 0 | 0 |
| hsa-miR-200a-3p | 0 | 1 | 2 |
| hsa-miR-200a-5p | 0 | 1 | 1 |
| hsa-miR-223-5p | 0 | 0 | 0 |
| hsa-miR-3064-5p | 0 | 0 | 1 |
| hsa-miR-3128 | 0 | 0 | 0 |
| hsa-miR-3131 | 0 | 0 | 0 |
| hsa-miR-3136-3p | 0 | 0 | 0 |
| hsa-miR-3138 | 0 | 1 | 1 |
| hsa-miR-3164 | 0 | 0 | 0 |
| hsa-miR-3176 | 0 | 1 | 2 |
| hsa-miR-3177-3p | 0 | 3 | 2 |
| hsa-miR-320a-5p | 0 | 0 | 1 |
| hsa-miR-3605-3p | 0 | 1 | 0 |
| hsa-miR-3609 | 0 | 0 | 0 |
| hsa-miR-3679-3p | 0 | 0 | 0 |
| hsa-miR-3688-5p | 0 | 0 | 0 |
| hsa-miR-3691-5p | 0 | 0 | 1 |
| hsa-miR-376c-5p | 0 | 0 | 0 |
| hsa-miR-3910 | 0 | 0 | 0 |
| hsa-miR-3940-5p | 0 | 0 | 1 |
| hsa-miR-4254 | 0 | 0 | 0 |
| hsa-miR-433-5p | 0 | 1 | 1 |
| hsa-miR-4488 | 0 | 0 | 0 |
| hsa-miR-4508 | 0 | 0 | 0 |
| hsa-miR-4517 | 0 | 0 | 0 |
| hsa-miR-4647 | 0 | 0 | 0 |
| hsa-miR-4665-5p | 0 | 2 | 1 |
| hsa-miR-4684-3p | 0 | 1 | 1 |
| hsa-miR-4715-5p | 0 | 0 | 0 |
| hsa-miR-4723-5p | 0 | 0 | 0 |
| hsa-miR-4728-3p | 0 | 0 | 0 |
| hsa-miR-4768-5p | 0 | 0 | 0 |
| hsa-miR-4797-5p | 0 | 0 | 0 |
| hsa-miR-494-5p | 0 | 2 | 1 |
| hsa-miR-5008-3p | 0 | 0 | 0 |
| hsa-miR-504-5p | 0 | 0 | 0 |
| hsa-miR-505-5p | 0 | 0 | 1 |
| hsa-miR-548k | 0 | 0 | 2 |
| hsa-miR-550a-3p | 0 | 0 | 1 |
| hsa-miR-550b-2-5p | 0 | 0 | 1 |
| hsa-miR-581 | 0 | 0 | 0 |
| hsa-miR-589-3p | 0 | 0 | 0 |
| hsa-miR-628-3p | 0 | 0 | 0 |
| hsa-miR-6501-5p | 0 | 0 | 0 |
| hsa-miR-6513-5p | 0 | 0 | 0 |
| hsa-miR-655-5p | 0 | 0 | 0 |
| hsa-miR-6741-5p | 0 | 0 | 0 |
| hsa-miR-6746-3p | 0 | 0 | 0 |
| hsa-miR-6758-5p | 0 | 0 | 0 |
| hsa-miR-6786-3p | 0 | 0 | 0 |
| hsa-miR-6806-3p | 0 | 0 | 0 |
| hsa-miR-6815-5p | 0 | 0 | 0 |
| hsa-miR-6875-3p | 0 | 0 | 0 |
| hsa-miR-6894-5p | 0 | 0 | 0 |
| hsa-miR-7113-5p | 0 | 0 | 1 |
| hsa-miR-7114-5p | 0 | 0 | 0 |
| hsa-miR-760 | 0 | 2 | 0 |
| hsa-miR-770-5p | 0 | 0 | 0 |
| hsa-miR-8072 | 0 | 0 | 0 |
| hsa-miR-889-5p | 0 | 1 | 0 |
| hsa-miR-933 | 0 | 0 | 0 |
| novel_13 | 0 | 1 | 0 |
| hsa-miR-103a-2-5p | 0 | 0 | 1 |
| hsa-miR-10523-5p | 0 | 0 | 0 |
| hsa-miR-11401 | 0 | 0 | 1 |
| hsa-miR-1228-5p | 0 | 1 | 0 |
| hsa-miR-1229-3p | 0 | 0 | 0 |
| hsa-miR-1234-3p | 0 | 0 | 0 |
| hsa-miR-1247-3p | 0 | 0 | 0 |
| hsa-miR-1270 | 0 | 0 | 0 |
| hsa-miR-1287-3p | 0 | 0 | 0 |
| hsa-miR-1295a | 0 | 0 | 0 |
| hsa-miR-1295b-3p | 0 | 0 | 0 |
| hsa-miR-133b | 0 | 0 | 0 |
| hsa-miR-135b-3p | 0 | 0 | 0 |
| hsa-miR-144-5p | 0 | 0 | 0 |
| hsa-miR-151b | 0 | 0 | 0 |
| hsa-miR-153-3p | 0 | 0 | 0 |
| hsa-miR-1909-3p | 0 | 0 | 1 |
| hsa-miR-194-3p | 0 | 0 | 0 |
| hsa-miR-197-5p | 0 | 1 | 0 |
| hsa-miR-200b-5p | 0 | 0 | 0 |
| hsa-miR-208b-3p | 0 | 0 | 0 |
| hsa-miR-20a-3p | 0 | 0 | 0 |
| hsa-miR-216a-5p | 0 | 0 | 0 |
| hsa-miR-301a-5p | 0 | 0 | 1 |
| hsa-miR-301b-3p | 0 | 0 | 0 |
| hsa-miR-302a-5p | 0 | 0 | 1 |
| hsa-miR-3059-5p | 0 | 0 | 0 |
| hsa-miR-3127-3p | 0 | 0 | 0 |
| hsa-miR-3134 | 0 | 0 | 0 |
| hsa-miR-3137 | 0 | 0 | 0 |
| hsa-miR-3139 | 0 | 0 | 0 |
| hsa-miR-3150a-5p | 0 | 0 | 0 |
| hsa-miR-3153 | 0 | 0 | 0 |
| hsa-miR-3154 | 0 | 0 | 0 |
| hsa-miR-3155b | 0 | 0 | 0 |
| hsa-miR-3157-3p | 0 | 0 | 0 |
| hsa-miR-3165 | 0 | 0 | 0 |
| hsa-miR-3166 | 0 | 0 | 0 |
| hsa-miR-3167 | 0 | 0 | 1 |
| hsa-miR-3170 | 0 | 0 | 0 |
| hsa-miR-3173-3p | 0 | 0 | 0 |
| hsa-miR-3175 | 0 | 0 | 1 |
| hsa-miR-3177-5p | 0 | 0 | 0 |
| hsa-miR-3187-5p | 0 | 1 | 0 |
| hsa-miR-3190-5p | 0 | 0 | 0 |
| hsa-miR-3191-3p | 0 | 0 | 0 |
| hsa-miR-3192-3p | 0 | 0 | 0 |
| hsa-miR-3200-5p | 0 | 0 | 0 |
| hsa-miR-33a-3p | 0 | 0 | 1 |
| hsa-miR-3611 | 0 | 0 | 0 |
| hsa-miR-3620-5p | 0 | 0 | 0 |
| hsa-miR-3622b-5p | 0 | 0 | 0 |
| hsa-miR-3659 | 0 | 0 | 0 |
| hsa-miR-3661 | 0 | 1 | 1 |
| hsa-miR-3675-3p | 0 | 0 | 1 |
| hsa-miR-3681-5p | 0 | 0 | 0 |
| hsa-miR-3685 | 0 | 0 | 0 |
| hsa-miR-375-3p | 0 | 0 | 0 |
| hsa-miR-376b-5p | 0 | 0 | 0 |
| hsa-miR-381-5p | 0 | 0 | 0 |
| hsa-miR-3917 | 0 | 1 | 0 |
| hsa-miR-3935 | 0 | 0 | 0 |
| hsa-miR-410-5p | 0 | 1 | 1 |
| hsa-miR-429 | 0 | 0 | 0 |
| hsa-miR-4423-5p | 0 | 0 | 0 |
| hsa-miR-4485-5p | 0 | 0 | 0 |
| hsa-miR-4524a-3p | 0 | 0 | 0 |
| hsa-miR-4524b-5p | 0 | 0 | 0 |
| hsa-miR-4529-5p | 0 | 0 | 0 |
| hsa-miR-454-5p | 0 | 1 | 2 |
| hsa-miR-4638-3p | 0 | 0 | 0 |
| hsa-miR-4649-5p | 0 | 0 | 0 |
| hsa-miR-4657 | 0 | 0 | 0 |
| hsa-miR-4661-5p | 0 | 0 | 1 |
| hsa-miR-4664-3p | 0 | 0 | 0 |
| hsa-miR-4667-5p | 0 | 0 | 0 |
| hsa-miR-4670-5p | 0 | 0 | 0 |
| hsa-miR-4690-5p | 0 | 0 | 0 |
| hsa-miR-4699-3p | 0 | 0 | 0 |
| hsa-miR-4707-3p | 0 | 0 | 0 |
| hsa-miR-4709-5p | 0 | 0 | 0 |
| hsa-miR-4731-3p | 0 | 0 | 0 |
| hsa-miR-4740-5p | 0 | 0 | 0 |
| hsa-miR-4741 | 0 | 1 | 0 |
| hsa-miR-4742-5p | 0 | 0 | 0 |
| hsa-miR-4754 | 0 | 0 | 0 |
| hsa-miR-4763-5p | 0 | 0 | 0 |
| hsa-miR-4768-3p | 0 | 0 | 0 |
| hsa-miR-4769-5p | 0 | 0 | 0 |
| hsa-miR-4803 | 0 | 0 | 0 |
| hsa-miR-497-3p | 0 | 0 | 0 |
| hsa-miR-5001-5p | 0 | 0 | 0 |
| hsa-miR-5091 | 0 | 0 | 0 |
| hsa-miR-509-3-5p | 0 | 0 | 0 |
| hsa-miR-5094 | 0 | 0 | 0 |
| hsa-miR-509-5p | 0 | 0 | 0 |
| hsa-miR-5100 | 0 | 0 | 0 |
| hsa-miR-511-5p | 0 | 0 | 0 |
| hsa-miR-5193 | 0 | 0 | 0 |
| hsa-miR-541-3p | 0 | 2 | 2 |
| hsa-miR-541-5p | 0 | 1 | 1 |
| hsa-miR-548ag | 0 | 0 | 0 |
| hsa-miR-548ao-3p | 0 | 1 | 0 |
| hsa-miR-548ay-3p | 0 | 0 | 1 |
| hsa-miR-552-3p | 0 | 0 | 0 |
| hsa-miR-5699-5p | 0 | 0 | 0 |
| hsa-miR-570-3p | 0 | 0 | 0 |
| hsa-miR-573 | 0 | 0 | 0 |
| hsa-miR-582-5p | 0 | 0 | 1 |
| hsa-miR-590-3p | 0 | 0 | 0 |
| hsa-miR-590-5p | 0 | 0 | 0 |
| hsa-miR-597-5p | 0 | 0 | 0 |
| hsa-miR-612 | 0 | 0 | 0 |
| hsa-miR-616-3p | 0 | 0 | 0 |
| hsa-miR-627-5p | 0 | 0 | 0 |
| hsa-miR-642a-5p | 0 | 0 | 0 |
| hsa-miR-642b-3p | 0 | 0 | 0 |
| hsa-miR-6511b-5p | 0 | 1 | 0 |
| hsa-miR-6516-5p | 0 | 0 | 0 |
| hsa-miR-652-5p | 0 | 0 | 0 |
| hsa-miR-6720-5p | 0 | 0 | 0 |
| hsa-miR-6724-5p | 0 | 2 | 1 |
| hsa-miR-6732-3p | 0 | 0 | 1 |
| hsa-miR-6732-5p | 0 | 0 | 0 |
| hsa-miR-6733-5p | 0 | 0 | 0 |
| hsa-miR-6735-3p | 0 | 0 | 0 |
| hsa-miR-6735-5p | 0 | 0 | 0 |
| hsa-miR-6737-3p | 0 | 0 | 0 |
| hsa-miR-6740-3p | 0 | 0 | 0 |
| hsa-miR-6753-3p | 0 | 1 | 1 |
| hsa-miR-676-5p | 0 | 0 | 0 |
| hsa-miR-6799-3p | 0 | 0 | 0 |
| hsa-miR-6802-3p | 0 | 0 | 0 |
| hsa-miR-6804-5p | 0 | 0 | 0 |
| hsa-miR-6809-5p | 0 | 0 | 0 |
| hsa-miR-6826-5p | 0 | 0 | 0 |
| hsa-miR-6839-5p | 0 | 0 | 0 |
| hsa-miR-6869-5p | 0 | 0 | 0 |
| hsa-miR-6873-3p | 0 | 0 | 0 |
| hsa-miR-6881-3p | 0 | 0 | 0 |
| hsa-miR-6891-3p | 0 | 0 | 0 |
| hsa-miR-7111-3p | 0 | 0 | 0 |
| hsa-miR-7155-3p | 0 | 0 | 0 |
| hsa-miR-767-3p | 0 | 0 | 0 |
| hsa-miR-7845-5p | 0 | 0 | 0 |
| hsa-miR-8485 | 0 | 0 | 0 |
| hsa-miR-937-5p | 0 | 0 | 0 |
| hsa-miR-98-3p | 0 | 0 | 0 |
| novel_12 | 0 | 0 | 1 |
| hsa-miR-10393-3p | 0 | 0 | 0 |
| hsa-miR-10394-5p | 0 | 0 | 0 |
| hsa-miR-10396a-5p | 0 | 0 | 0 |
| hsa-miR-10398-5p | 0 | 0 | 0 |
| hsa-miR-10399-3p | 0 | 0 | 0 |
| hsa-miR-10399-5p | 0 | 0 | 0 |
| hsa-miR-10526-3p | 0 | 1 | 0 |
| hsa-miR-122-3p | 0 | 0 | 0 |
| hsa-miR-122-5p | 0 | 0 | 0 |
| hsa-miR-1226-5p | 0 | 0 | 0 |
| hsa-miR-1229-5p | 0 | 0 | 0 |
| hsa-miR-122b-3p | 0 | 0 | 0 |
| hsa-miR-1237-3p | 0 | 0 | 0 |
| hsa-miR-124-3p | 0 | 1 | 1 |
| hsa-miR-1250-5p | 0 | 1 | 0 |
| hsa-miR-1265 | 0 | 0 | 0 |
| hsa-miR-126-5p | 0 | 0 | 0 |
| hsa-miR-1267 | 0 | 0 | 0 |
| hsa-miR-1269a | 0 | 0 | 0 |
| hsa-miR-1275 | 0 | 0 | 0 |
| hsa-miR-1292-3p | 0 | 0 | 0 |
| hsa-miR-1298-5p | 0 | 3 | 7 |
| hsa-miR-133a-5p | 0 | 0 | 0 |
| hsa-miR-135a-5p | 0 | 0 | 0 |
| hsa-miR-146a-3p | 0 | 0 | 2 |
| hsa-miR-149-3p | 0 | 1 | 0 |
| hsa-miR-150-5p | 0 | 0 | 0 |
| hsa-miR-1538 | 0 | 0 | 0 |
| hsa-miR-181d-3p | 0 | 0 | 0 |
| hsa-miR-183-3p | 0 | 0 | 0 |
| hsa-miR-183-5p | 0 | 8 | 13 |
| hsa-miR-188-5p | 0 | 0 | 1 |
| hsa-miR-1908-3p | 0 | 0 | 0 |
| hsa-miR-190b-5p | 0 | 0 | 0 |
| hsa-miR-1910-3p | 0 | 0 | 0 |
| hsa-miR-1910-5p | 0 | 2 | 1 |
| hsa-miR-1913 | 0 | 0 | 0 |
| hsa-miR-1914-5p | 0 | 0 | 0 |
| hsa-miR-203a-3p | 0 | 0 | 0 |
| hsa-miR-203b-5p | 0 | 0 | 0 |
| hsa-miR-204-3p | 0 | 0 | 0 |
| hsa-miR-205-5p | 0 | 0 | 0 |
| hsa-miR-215-5p | 0 | 0 | 1 |
| hsa-miR-217-3p | 0 | 0 | 0 |
| hsa-miR-2276-3p | 0 | 1 | 0 |
| hsa-miR-2682-3p | 0 | 0 | 1 |
| hsa-miR-26a-1-3p | 0 | 0 | 0 |
| hsa-miR-301a-3p | 0 | 0 | 0 |
| hsa-miR-301b-5p | 0 | 1 | 0 |
| hsa-miR-302a-3p | 0 | 0 | 0 |
| hsa-miR-302b-3p | 0 | 0 | 1 |
| hsa-miR-302c-3p | 0 | 0 | 0 |
| hsa-miR-302d-3p | 0 | 0 | 0 |
| hsa-miR-3065-3p | 0 | 1 | 0 |
| hsa-miR-3124-5p | 0 | 0 | 0 |
| hsa-miR-3126-3p | 0 | 0 | 0 |
| hsa-miR-3126-5p | 0 | 0 | 0 |
| hsa-miR-3146 | 0 | 0 | 0 |
| hsa-miR-3148 | 0 | 0 | 0 |
| hsa-miR-3150a-3p | 0 | 0 | 0 |
| hsa-miR-3150b-3p | 0 | 0 | 0 |
| hsa-miR-3150b-5p | 0 | 0 | 0 |
| hsa-miR-3152-3p | 0 | 0 | 0 |
| hsa-miR-3157-5p | 0 | 0 | 0 |
| hsa-miR-3163 | 0 | 0 | 0 |
| hsa-miR-3181 | 0 | 0 | 0 |
| hsa-miR-3183 | 0 | 0 | 0 |
| hsa-miR-3188 | 0 | 0 | 0 |
| hsa-miR-3190-3p | 0 | 0 | 0 |
| hsa-miR-3191-5p | 0 | 0 | 0 |
| hsa-miR-3192-5p | 0 | 0 | 0 |
| hsa-miR-3198 | 0 | 0 | 0 |
| hsa-miR-323b-5p | 0 | 0 | 0 |
| hsa-miR-329-5p | 0 | 1 | 1 |
| hsa-miR-338-5p | 0 | 0 | 0 |
| hsa-miR-346 | 0 | 0 | 0 |
| hsa-miR-3529-5p | 0 | 0 | 0 |
| hsa-miR-362-3p | 0 | 0 | 0 |
| hsa-miR-363-3p | 0 | 0 | 0 |
| hsa-miR-3652 | 0 | 0 | 0 |
| hsa-miR-3675-5p | 0 | 1 | 1 |
| hsa-miR-3678-3p | 0 | 0 | 0 |
| hsa-miR-3678-5p | 0 | 0 | 0 |
| hsa-miR-3680-3p | 0 | 0 | 0 |
| hsa-miR-3680-5p | 0 | 0 | 0 |
| hsa-miR-383-5p | 0 | 1 | 0 |
| hsa-miR-3907 | 0 | 0 | 0 |
| hsa-miR-3918 | 0 | 0 | 0 |
| hsa-miR-3929 | 0 | 0 | 0 |
| hsa-miR-3939 | 0 | 0 | 0 |
| hsa-miR-3944-5p | 0 | 0 | 0 |
| hsa-miR-412-3p | 0 | 0 | 1 |
| hsa-miR-4423-3p | 0 | 0 | 0 |
| hsa-miR-4425 | 0 | 0 | 0 |
| hsa-miR-4426 | 0 | 0 | 0 |
| hsa-miR-4435 | 0 | 1 | 1 |
| hsa-miR-4440 | 0 | 0 | 0 |
| hsa-miR-4467 | 0 | 0 | 0 |
| hsa-miR-4470 | 0 | 0 | 0 |
| hsa-miR-4479 | 0 | 0 | 0 |
| hsa-miR-448 | 0 | 0 | 0 |
| hsa-miR-4489 | 0 | 0 | 0 |
| hsa-miR-4498 | 0 | 0 | 0 |
| hsa-miR-449b-3p | 0 | 0 | 0 |
| hsa-miR-4504 | 0 | 0 | 1 |
| hsa-miR-4506 | 0 | 0 | 0 |
| hsa-miR-4522 | 0 | 0 | 0 |
| hsa-miR-4636 | 0 | 0 | 0 |
| hsa-miR-4638-5p | 0 | 0 | 0 |
| hsa-miR-4642 | 0 | 0 | 0 |
| hsa-miR-4669 | 0 | 0 | 0 |
| hsa-miR-4672 | 0 | 0 | 0 |
| hsa-miR-4674 | 0 | 0 | 0 |
| hsa-miR-4676-3p | 0 | 0 | 0 |
| hsa-miR-4676-5p | 0 | 0 | 0 |
| hsa-miR-4677-5p | 0 | 0 | 0 |
| hsa-miR-4681 | 0 | 0 | 0 |
| hsa-miR-4682 | 0 | 0 | 0 |
| hsa-miR-4685-3p | 0 | 0 | 0 |
| hsa-miR-4687-3p | 0 | 0 | 0 |
| hsa-miR-4688 | 0 | 0 | 0 |
| hsa-miR-4691-3p | 0 | 0 | 0 |
| hsa-miR-4695-3p | 0 | 0 | 0 |
| hsa-miR-4706 | 0 | 0 | 0 |
| hsa-miR-4707-5p | 0 | 0 | 0 |
| hsa-miR-4709-3p | 0 | 0 | 0 |
| hsa-miR-4713-3p | 0 | 0 | 0 |
| hsa-miR-4717-3p | 0 | 0 | 0 |
| hsa-miR-4717-5p | 0 | 0 | 0 |
| hsa-miR-4727-3p | 0 | 0 | 0 |
| hsa-miR-4731-5p | 0 | 0 | 0 |
| hsa-miR-4742-3p | 0 | 0 | 0 |
| hsa-miR-4743-5p | 0 | 0 | 0 |
| hsa-miR-4744 | 0 | 0 | 0 |
| hsa-miR-4745-5p | 0 | 0 | 0 |
| hsa-miR-4761-5p | 0 | 0 | 0 |
| hsa-miR-4769-3p | 0 | 0 | 0 |
| hsa-miR-4784 | 0 | 0 | 0 |
| hsa-miR-4785 | 0 | 0 | 0 |
| hsa-miR-4799-5p | 0 | 0 | 0 |
| hsa-miR-490-5p | 0 | 23 | 0 |
| hsa-miR-495-5p | 0 | 0 | 1 |
| hsa-miR-4999-5p | 0 | 0 | 0 |
| hsa-miR-5000-3p | 0 | 0 | 0 |
| hsa-miR-5004-5p | 0 | 0 | 0 |
| hsa-miR-5009-5p | 0 | 0 | 0 |
| hsa-miR-502-5p | 0 | 0 | 0 |
| hsa-miR-5088-5p | 0 | 0 | 0 |
| hsa-miR-514a-3p | 0 | 0 | 0 |
| hsa-miR-5187-5p | 0 | 0 | 0 |
| hsa-miR-548a-3p | 0 | 0 | 0 |
| hsa-miR-548ak | 0 | 0 | 0 |
| hsa-miR-548am-3p | 0 | 0 | 0 |
| hsa-miR-548ap-5p | 0 | 0 | 0 |
| hsa-miR-548ar-3p | 0 | 0 | 0 |
| hsa-miR-548av-3p | 0 | 0 | 0 |
| hsa-miR-548d-3p | 0 | 0 | 0 |
| hsa-miR-548q | 0 | 0 | 0 |
| hsa-miR-548s | 0 | 0 | 0 |
| hsa-miR-551a | 0 | 0 | 0 |
| hsa-miR-551b-5p | 0 | 2 | 0 |
| hsa-miR-5582-3p | 0 | 0 | 0 |
| hsa-miR-5699-3p | 0 | 0 | 0 |
| hsa-miR-584-3p | 0 | 0 | 0 |
| hsa-miR-598-5p | 0 | 0 | 0 |
| hsa-miR-605-3p | 0 | 0 | 1 |
| hsa-miR-6132 | 0 | 0 | 0 |
| hsa-miR-624-5p | 0 | 0 | 0 |
| hsa-miR-642a-3p | 0 | 0 | 1 |
| hsa-miR-642b-5p | 0 | 0 | 1 |
| hsa-miR-643 | 0 | 0 | 0 |
| hsa-miR-6509-3p | 0 | 0 | 0 |
| hsa-miR-6509-5p | 0 | 0 | 0 |
| hsa-miR-6511a-5p | 0 | 0 | 0 |
| hsa-miR-6514-5p | 0 | 0 | 0 |
| hsa-miR-656-5p | 0 | 0 | 0 |
| hsa-miR-663a | 0 | 0 | 0 |
| hsa-miR-664b-5p | 0 | 0 | 0 |
| hsa-miR-6726-3p | 0 | 0 | 0 |
| hsa-miR-6726-5p | 0 | 0 | 0 |
| hsa-miR-6727-5p | 0 | 0 | 0 |
| hsa-miR-6729-5p | 0 | 0 | 0 |
| hsa-miR-6736-5p | 0 | 0 | 0 |
| hsa-miR-6739-3p | 0 | 0 | 0 |
| hsa-miR-6741-3p | 0 | 0 | 0 |
| hsa-miR-6747-3p | 0 | 0 | 0 |
| hsa-miR-6751-5p | 0 | 0 | 0 |
| hsa-miR-6756-5p | 0 | 0 | 0 |
| hsa-miR-6757-5p | 0 | 0 | 0 |
| hsa-miR-6759-3p | 0 | 0 | 0 |
| hsa-miR-6761-5p | 0 | 0 | 0 |
| hsa-miR-6764-3p | 0 | 0 | 0 |
| hsa-miR-6764-5p | 0 | 0 | 0 |
| hsa-miR-6766-3p | 0 | 0 | 0 |
| hsa-miR-6766-5p | 0 | 0 | 0 |
| hsa-miR-6775-5p | 0 | 0 | 0 |
| hsa-miR-6779-3p | 0 | 0 | 0 |
| hsa-miR-6780a-5p | 0 | 0 | 0 |
| hsa-miR-6780b-3p | 0 | 0 | 0 |
| hsa-miR-6783-3p | 0 | 0 | 0 |
| hsa-miR-6789-3p | 0 | 0 | 0 |
| hsa-miR-6795-5p | 0 | 0 | 0 |
| hsa-miR-6796-3p | 0 | 0 | 0 |
| hsa-miR-6797-3p | 0 | 0 | 0 |
| hsa-miR-6800-3p | 0 | 0 | 0 |
| hsa-miR-6802-5p | 0 | 0 | 0 |
| hsa-miR-6803-3p | 0 | 0 | 0 |
| hsa-miR-6805-5p | 0 | 0 | 0 |
| hsa-miR-6808-3p | 0 | 0 | 0 |
| hsa-miR-6810-3p | 0 | 0 | 0 |
| hsa-miR-6812-5p | 0 | 0 | 0 |
| hsa-miR-6817-3p | 0 | 0 | 0 |
| hsa-miR-6817-5p | 0 | 0 | 0 |
| hsa-miR-6818-5p | 0 | 0 | 0 |
| hsa-miR-6819-3p | 0 | 0 | 0 |
| hsa-miR-6819-5p | 0 | 0 | 1 |
| hsa-miR-6820-5p | 0 | 0 | 0 |
| hsa-miR-6824-3p | 0 | 0 | 0 |
| hsa-miR-6827-3p | 0 | 0 | 0 |
| hsa-miR-6829-5p | 0 | 0 | 0 |
| hsa-miR-6830-5p | 0 | 0 | 0 |
| hsa-miR-6834-3p | 0 | 0 | 0 |
| hsa-miR-6837-3p | 0 | 1 | 0 |
| hsa-miR-6840-5p | 0 | 0 | 1 |
| hsa-miR-6842-5p | 0 | 0 | 0 |
| hsa-miR-6845-5p | 0 | 0 | 0 |
| hsa-miR-6849-5p | 0 | 0 | 0 |
| hsa-miR-6853-3p | 0 | 0 | 0 |
| hsa-miR-6857-3p | 0 | 0 | 0 |
| hsa-miR-6858-5p | 0 | 0 | 0 |
| hsa-miR-6859-3p | 0 | 0 | 0 |
| hsa-miR-6859-5p | 0 | 0 | 0 |
| hsa-miR-6862-5p | 0 | 0 | 0 |
| hsa-miR-6870-3p | 0 | 0 | 0 |
| hsa-miR-6871-3p | 0 | 0 | 0 |
| hsa-miR-6882-5p | 0 | 0 | 0 |
| hsa-miR-6883-3p | 0 | 0 | 0 |
| hsa-miR-6886-5p | 0 | 0 | 0 |
| hsa-miR-6889-5p | 0 | 0 | 0 |
| hsa-miR-6894-3p | 0 | 0 | 0 |
| hsa-miR-7108-5p | 0 | 0 | 0 |
| hsa-miR-7110-3p | 0 | 0 | 0 |
| hsa-miR-7156-5p | 0 | 0 | 0 |
| hsa-miR-7-2-3p | 0 | 0 | 0 |
| hsa-miR-758-5p | 0 | 0 | 0 |
| hsa-miR-7854-3p | 0 | 0 | 0 |
| hsa-miR-873-5p | 0 | 0 | 0 |
| hsa-miR-877-3p | 0 | 1 | 1 |
| hsa-miR-935 | 0 | 27 | 20 |
| hsa-miR-9-3p | 0 | 0 | 0 |
| hsa-miR-96-5p | 0 | 1 | 1 |
| novel_15 | 0 | 0 | 0 |
| novel_16 | 0 | 0 | 0 |
| novel_4 | 0 | 0 | 0 |

***

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### REFERENCES

1. Witwer K W, Buzás E I, Bemis L T, et al. Standardization of sample collection, isolation and analysis methods in extracellular vesicle research. J Extracell Vesicles 2013; 2. doi: 10.3402/jev.v2i0.20360.
2. Xu R, Greening D W, Zhu H J, et al. Extracellular vesicle isolation and characterization: toward clinical application. J Clin Invest 2016; 126:1152-1162. doi: 10.1172/JCI81129.
3. El Andaloussi S, Mäger I, Breakefield X O, Wood M J A. Extracellular vesicles: biology and emerging therapeutic opportunities. Nat Rev Drug Discov. 2013; 12:347-357. doi: 10.1038/nrd3978.
4. Théry C, Amigorena S, Raposo G, Clayton A. Isolation and characterization of exosomes from cell culture supernatants and biological fluids. Curr Protoc Cell Biol. Editor. Board Juan Bonifacino Al (2006) Chapter 3, Unit 3.22. doi: 10.1002/0471143030.cb0322s30.
5. Abramowicz A, Widlak P, Pietrowska M. Proteomic analysis of exosomal cargo: the challenge of high purity vesicle isolation. Mol Biosyst 2016; 12:1407-1419. doi: 10.1039/c6mb00082g.
6. Van Deun J, Mestdagh P, Sormunen R, et al. The impact of disparate isolation methods for extracellular vesicles on downstream RNA profiling. J Extracell Vesicles 2014) 3. doi: 10.3402/jev.v3.24858.
7. Webber J, Clayton A. How pure are your vesicles? J Extracell Vesicles 2013; 2. doi: 10.3402/jev.v2i0.19861.
8. Mathivanan S, Simpson R J. ExoCarta: A compendium of exosomal proteins and RNA. Proteomics 2009; 9:4997-5000. doi: 10.1002/pmic.200900351.
9. Hematology 4th edition. Williams W J, Beutler E, Erslev A J, Lichtman M A (eds). McGraw-Hill Inc, New York, 1990.
10. Kay H E M. How many cell-generations? The Lancet 1995; August 28.
11. Mintz B, Russell E S. Gene induced embryological modifications of primordial germ cells in the mouse. J Exp Zool 1957; 134:207-237.
12. Weiss L. The hematopoietic microenvironment of the bone marrow: an ultrastructural study of the stroma in rats. Anatom Rec 1976; 186: 161.
13. Westen H, Bainton D F. Association of alkaline phosphatase-positive reticulum cells in bone marrow with granulocyte precursors. J Exp Med 1979; 150: 919.
14. Lichtman M A. The ultrastructure of the hematopoietic microenvironment of the marrow: a review. Exp Hematol 1981; 9: 391.
15. Bianco P, Riminucci M. The bone marrow stroma in vivo: ontogeny, structure, cellular composition and changes in disease. In: Beresford J N, Owens M E, editors. Marrow stromal cell culture. Handbooks in Practival Animal Cell Biology. Cambridge, UK: Cambridge University Press, 1998; p 1025.
16. Dexter T M. Stromal cell associated haemopoiesis. J Cell Physiol Suppl 1982; 1: 87.
17. Friedenstein A J. Precursor cells of mechanocyte. Int Rev Cytol 1976; 47: 327.
18. Quirici N, Soligo D, Bossolasco P, et al. Isolation of bone marrow mesenchymal stem cells by anti-nerve growth factor receptor antibodies. Exp Hematol 2002; 30(7): 783.
19. Jones E, English A, Kinsey S E, et al. Optimization of a flow cytometry-based protocol for detection and phenotypic characterization of multipotent mesenchymal stromal cells from human bone marrow. Cytometry B Clin Cytom 2006; 70: 391-399.
20. Zannettino A, Paton S, Kortesidis A, et al. Human multipotential mesenchymal/stromal stem cell are derived from a discrete subpopulation of STRO-1^{bright}/CD34-/CD45-/glycophorin-A-bone marrow cells. Haematogica 2007; 92(12): 1707.
21. Dominici M, Le Blanc K, Mueller I, et al. Minimal criteria for defining multipotent mesenchymal stromal cell. The International Society of Cellular Therapy position statement. Cytotherapy 2006; 8(4): 315.
22. Freidenstein A J, Gorskaja J F, Kilagina N N. Fibroblast precursors in normal and irradiated hematopoietic organs. Exp Hematol 1976; 4(5): 267-74.
23. Le Blanc K, Tammik C, Rosendahl K, et al. HLA expression expression and immunologic properties of differentiated and undifferentiated mesenchymal stem cells. Exp Hematol 2003; 31(10): 890-6.
24. Klyushnenkova E, Shustova V, Mosca J, et al. Human mesenchymal stem cells induce unresponsiveness in preactivated but not naïve alloantigen specific T cells. Exp Hematol 1999; 27: abstract 122.
25. Klyushnenkova E, Mosa J D, Zernetkina V, et al. T cell responses to allogeneic human mesenchymal stem cells: immunogenicity, tolerance, and suppression. J Biomed Sci 2005; 12(1): 47-57.
26. Le Blanc K, Ringden O. Immunobiology of human mesenchymal stem cells and future use in hematopoietic stem cell transplantation. Biol Blood Marrow Transplant 2005; 11(5): 321-34.
27. Le Blanc K, Rasmusson I, Gotherstrom C, et al. Mesenchymal stem cells inhibit the expression of CD25 (interleukin-2 receptor) and CD38 on phytohaemagglutinin-activated lymphocytes. Scand J Immunol 2004; 60(3): 307-15.
28. Bartholomew A, Sturgeon C, Siatskas M, et al. Mesenchymal stem cells suppress lymphocyte proliferation in vitro and prolong skin graft survival in vivo. Exp Hematol 2002; 30(1): 42-8.
29. Lazarus H M, Koc O N, Devine S M, et al. Cotransplantation of HLA-identical sibling culture-expanded mesenchymal stem cells and hematopoietic stem cells in hematologic malignancy patients. Biol Blood Marrow Transplant 2005; 11(5): 389-98.
30. Aggarwal S, Pittenger M F. Human mesenchymal stem cells modulate allogeneic immune cell responses. Blood 2005; 105(4): 1815-22.
31. Lietchy K W, MacKenzie T C, Shaaban A F, et al. Human mesenchymal stem cells engraft and demonstrate site-specific differentiation after in utero transplantation in sheep. Nat Med 2000; 6(11): 1282-6.
32. Grinnemo K H, Mansson A, Dellgren G, et al. Xenoreactivity and engraftment of human mesenchymal stem cells transplanted into infracted rat myocardium. J Thorac Cardiovasc Surg 2004; 127(5): 1293-300.
33. Arinzeh T L, Peter S L, Archambault M P, et al. Allogeneic mesenchymal stem cells regenerate bone in a critical-sized canine segmental defect. J Bone Joint Surg Am 2003; 85-A(10): 1927-35.
34. Murphy J M, Fink D J, Hunziker E B, Barry F P. Stem cells therapy in a caprine model of osteoarthritis. Arthritis Rheum 2003; 48(12): 3464-74.
35. Mahmud N, Pang W, Cobbs C, et al. Studies on the route of administration and role of conditioning with radiation on unrelated allogeneic mismatched mesenchymal stem cell engraftment in a nonhuman primate model. Exp Hematol 2004; 32(5): 494-501.
36. Haylock D N, Nilsson S K. Stem cell regulation by the hematopoietic stem cell niche. Cell Cycle 2008; 4(10): 1353-5.
37. Weiss L. The hematopoietic microenvironment of the bone marrow: an ultrastructural study of the stroma in rats. Anatom Rec 1976; 186: 161.
38. Decker C, Greggs R, Duggan K, et al. Adhesive multiplicity in the interaction of embryonic fibroblasts and myoblasts with extracellular matrices. J Cell Biol 1984; 99: 1398.
39. Choy M, Oltjen S L, Otani Y S, et al. Fibroblast growth factor-2 stimulates embryonic cardiac mesenchymal cell proliferation. Devel Dynamics 1996; 206: 193.
40. Baudino T A, Carver W, Giles W, Borg T K. Cardiac fibroblasts: friend or foe? Am J Physiol Heart Circ Physiol 2006; 291: H1015.
41. Mazhari R, Hare J M. Mechanisms of action of mesenchymal stem cells in cardiac repair: potential influences on the cardiac stem cell niche. Nat Clin Pract Cardiovasc Med 2007; 4 suppl 1: S21-6.
42. Weiss L. The hematopoietic microenvironment of the bone marrow: an ultrastructural study of the stroma in rats. Anatom Rec 1976; 186: 161.
43. Westen H, Bainton D F. Association of alkaline phosphatase-positive reticulum cells in bone marrow with granulocyte precursors. J Exp Med 1979; 150: 919.
44. Lichtman M A. The ultrastructure of the hematopoietic microenvironment of the marrow: a review. Exp Hematol 1981; 9: 391.
45. Bianco P, Riminucci M. The bone marrow stroma in vivo: ontogeny, structure, cellular composition and changes in disease. In: Beresford J N, Owens M E, editors. Marrow stromal cell culture. Handbooks in Practival Animal Cell Biology. Cambridge, UK: Cambridge University Press, 1998; p 1025.
46. Dexter T M. Stromal cell associated haemopoiesis. J Cell Physiol Suppl 1982; 1: 87.
47. Russell E S. Hereditary anemias of the mouse: a review for geneticists. Adv Genet 1979, 20:357-459.
48. Beltrami A P, Cessili D. Multipotnet cells can be generated in vitro from several adult human organs (heart, liver, and bone marrow). Blood 2007,110(9):3438-3446
49. Urbanek K, Cesselli D, Rota M. Stem cell niches in the adult mouse heart. Proc Natl Acad Sci USA 2006, 103(24):9226-9231.
50. Vandesompele, J., De Preter K, Pattyn F, et al. Accurate normalization of real-time quantitative R T-PCR data by geometric averaging of multiple internal control genes. Genome Biol, 2002. 3(7): p. RESEARCH0034.
51. Townley-Tilson W H D, Callis T E, Wang D Z. MicroRNAs 1, 133 and 206: Critical factors of skeletal and cardiac muscle development, function, and disease. Int J Biochem Cell Biol 2009, In Press.
52. Cordes K R, Srivastava D. MicroRNA regulation of cardiovascular development. Cir Res 2009, 104:724-732.
53. Luzi E, Marini F, Sala S C, et al. Osteogenic differentiation of human adipose tissue-derived stem cells is modulated by the miR-26a targeting of the SMAD1 transcription factor. Bone Miner Res 2008, 23(2):287-295.
54. Foshay K M, Gallicano G I. miR-17 family miRNAs are expressed during early mammalian development and regulate stem cell differentiation. Dev Biol 2009, 326(2):431-443.
55. McCarthy J J. MicroRNA-206: The skeletal muscle specific myomiR. Biochima et Biophysica Acta 2008, 1779:682-691.
56. Anderson C, Catoe H, Werner R. MIR-206 regulates connexin 43 expression during skeletal muscle development. Nucleic Acids Research 2006, 34(20):5863-5871.
57. Shan Z X, Lin Q X, Fu Y H, et al. Upregulated expression of miR-1/miR-206 in a rat model of myocardial infarction. Biochemical and Biophysical Research Communications 2009, 381:597-601.
58. Kidd S, Spaeth E, Watson K, et al. Origins of the tumor microenvironment: Quantitative assessment of adipose-derived and bone marrow-derived stroma. PLoS ONE 2012, 7(2):1-12.
59. Leia M J, Shah D J, reardon M J. Primary cardiac tumors. Tex Heart Inst J 2011, 38(3):261-262.
60. Negrin M, Calin G A. Breast cancer metastitis: a micro RNA story. Breast Cancer Research 2008, 10:303.
61. Adams B D, Cowee D M, White B A. The role of miR-206 in the epidermal growth factor (EGF) induced repression of estrogen receptor-alpha (ERalpha) signaling and luminal phenotype in MCF-7 breast cancer cells. Mol Endocrinol 2009, 23(8):1215-1230.
62. Taulli R, Bersani F, Foglizzo, et al. The muscle-specific microRNA miR-206 blocks human rhabdomyosarcoma growth in xenotransplanted mice by promoting myogenic differentiation. J Clin Invest 2009, 119:2366-2378.
63. Zhou L, et al. Integrated Profiling of MicroRNAs and mRNAs: MicroRNAs Located on Xq27.3 Associate with Clear Cell Renal Cell Carcinoma, PlosOne, 2010. https://doi.org/10.1371/journal.pone.0015224

What is claimed is:

1. A composition comprising an exosome derived from a fetal cardiac stromal cell, said exosome containing one or more of miR1, miR133a, and miR206.

2. The composition of claim 1, wherein said exosome contains at least two of miR1, miR133a, and miR206.

3. The composition of claim 1, wherein said exosome contains all three of miR1, miR133a, and miR206.

4. A method of treating a patient suffering from a cardiac disease, disorder or injury, said method comprising administering the exosome of claim 1 to the patient's cardiac tissue.

5. The method of claim 4, wherein the patient's cardiac tissue comprises infarcted myocardial tissue.

6. A method of treating a cardiac disease, disorder or injury in a subject, the method comprising administering to a patient in need thereof, an effective amount of a composition comprising an agent comprising an exosome containing one or more of miR1, miR133a, and miR206.

7. The method of claim 6, wherein said exosome contains at least two of miR1, miR133a, and miR206.

8. The method of claim 6, wherein said exosome contains all three of miR1, miR133a, and miR206.

9. The method of claim 6, wherein the cardiac disease, disorder or injury is myocardial infarction.

10. The method of claim 6, wherein the composition is administered to the patient by a route selected from the group consisting of local, topical, subcutaneous, intravenous, oral, intramuscular, and combinations thereof.

11. The method of claim 6, wherein the composition is administered to the patient by myocardial injection.

12. A method for producing a therapeutic composition comprising:
harvesting a population of exosomes from fetal cardiac stromal cell, said exosomes containing one or more of miR1, miR133a, and miR206.

13. The method of claim 12, wherein said exosome contains at least two of miR1, miR133a, and miR206.

14. The method of claim 12, wherein said exosome contains all three of miR1, miR133a, and miR206.

15. A composition comprising an exosome derived from a fetal stromal cell, said exosome containing two or more of miR which are more than two fold TPM in the fetal stromal cell than in an adult mesenchymal stem cell, and where said exosome is targeted for a reparative therapeutic indication towards a degenerative tissue disease that is specific to the tissue from which said fetal stromal cell is derived.

16. The composition of claim 15, where the exosomes are derived from fetal cardiac stromal cells.

17. The composition of claim 15, wherein the two or more miR are selected from the set of miR1, miR133a, miR206, hsa-miR-146a-5p, hsa-miR-490-3p, hsa-miR-9-5p, hsa-miR-3117-3p, hsa-miR-4521, hsa-miR-412-5p, hsa-miR-541-3p, hsa-miR-6724-5p, hsa-miR-182-5p, hsa-miR-454-5p, hsa-miR-206, hsa-miR-584-5p, hsa-miR-7706, hsa-miR-3177-3p, hsa-miR-410-5p, hsa-miR-541-5p, hsa-miR-3175, hsa-miR-204-5p, hsa-miR-3661, hsa-miR-302a-5p, hsa-miR-4661-5p, hsa-miR-543, hsa-miR-103a-2-5p, hsa-miR-3176, hsa-miR-433-5p, hsa-miR-10a-5p, hsa-miR-6753-3p, hsa-miR-330-5p, hsa-miR-11401, hsa-miR-582-3p, hsa-miR-2355-3p, hsa-miR-6511b-5p, hsa-miR-494-5p, hsa-miR-548k, hsa-miR-200a-3p, hsa-miR-744-3p, hsa-miR-487a-3p, hsa-miR-4665-5p, hsa-miR-598-3p, hsa-miR-548ao-3p, hsa-miR-301a-5p, hsa-miR-940, hsa-miR-323a-5p, hsa-miR-1228-5p, hsa-miR-760, hsa-miR-495-3p hsa-miR-937-3p, hsa-miR-4684-3p, hsa-miR-337-3p, hsa-miR-3138, hsa-miR-433-3p, hsa-miR-487b-5p, hsa-miR-2682-5p, hsa-miR-6732-3p, hsa-miR-3167, hsa-miR-3187-3p, hsa-miR-219a-1-3p, hsa-miR-18a-3p, hsa-miR-1343-3p, hsa-miR-98-5p, hsa-miR-191-3p, hsa-miR-33a-3p, hsa-miR-143-3p, hsa-miR-432-5p, hsa-miR-548ay-3p, hsa-miR-1307-3p, hsa-miR-8485, hsa-miR-487a-5p, hsa-miR-451a, hsa-miR-33b-3p, hsa-miR-3155b, hsa-miR-380-5p, hsa-miR-10401-3p, hsa-miR-539-5p hsa-miR-323b-3p, hsa-miR-3605-3p, hsa-miR-3064-5p, hsa-miR-3691-5p, hsa-miR-6827-5p, hsa-miR-487b-3p, hsa-miR-3074-5p, hsa-miR-100-5p, hsa-miR-24-3p, hsa-miR-149-5p, hsa-miR-1909-3p, hsa-miR-3675-3p, hsa-miR-323a-3p, hsa-miR-129-5p, hsa-miR-187-3p, hsa-miR-431-5p, and hsa-miR-200a-5p.

18. The composition of claim 15, wherein the two or more miR are selected to treat a clinical indication selected from the set of heart failure, myocardial infarction, and chronic myocardial ischemia.

19. The composition of claim 15, where the exosomes are derived from fetal liver stromal cells to treat liver disease that results from drug toxicity, alcoholism, Hepatitis B, or Hepatitis C.

20. The composition of claim 15, where the exosomes are derived from fetal brain stromal cells to treat stroke or Parkinson's disease.

21. The composition of claim 15, where the exosomes are derived from fetal kidney stromal cells to treat kidney failure.

22. The composition of claim 15, where the exosomes are derived from fetal skin stromal cells to treat psoriasis or other diseases and conditions of the skin.

23. The composition of claim 15, where the exosomes are derived from fetal lung stromal cells and used to treat asthma and emphysema.

24. The composition of claim 15, where the exosomes are derived from fetal pancreas stromal cells and used to treat type 1 diabetes.

25. The composition of claim 15, where the exosomes are derived from fetal pancreas stromal cells and used to treat type 2 diabetes.

26. The composition of claim 15 in which said exosome contains three or more of miR which are more than two fold TPM in the fetal stromal cell than in the adult mesenchymal stem cell, and where said exosome is targeted for a reparative therapeutic indication towards a degenerative tissue disease that is specific to the tissue from which said fetal stromal cell is derived.

27. The composition of claim 15 in which said exosome contains four or more of miR which are more than two fold TPM in the fetal stromal cell than in the adult mesenchymal stem cell, and where said exosome is targeted for a reparative therapeutic indication towards a degenerative tissue disease that is specific to the tissue from which said fetal stromal cell is derived.

28. The composition of claim 15 in which said exosome contains two or more of miR which are more than three fold TPM in the fetal stromal cell than in the adult mesenchymal stem cell, and where said exosome is targeted for a reparative therapeutic indication towards a degenerative tissue disease that is specific to the tissue from which said fetal stromal cell is derived.

29. A method of treating a patient suffering from the degenerative tissue disease, said method comprising administering the exosome of claim 15 to tissue of the patient.
